(19) United States
(12) Patent Application Publication
(10) Pub. No.: US 2026/0326172 A1
(43) Pub. Date: Oct. 22, 2026

(54) NOVEL MUNICIPAL SOLID WASTE PROCESSING SYSTEM FOR CONVERSION-READY FEEDSTOCKS FOR MATERIALS AND ENERGY RECOVERY

(71) Applicants: North Carolina State University, Raleigh, NC (US); Alliance for Sustainable Energy, LLC, Golden, CO (US)

(72) Inventors: Lokendra Pal, Cary, NC (US); Sachin Agate, Chicago, IL (US); Naimul Haque, Raleigh, NC (US); Lucian Lucia, Apex, NC (US); Richard A. Venditti, Apex, NC (US); Hasan Jameel, Raleigh, NC (US); Ashutosh Mittal, Golden, CO (US); John Michael Yarbrough, Golden, CO (US)

(73) Assignees: North Carolina State University, Raleigh, NC (US); Alliance for Sustainable Energy, LLC, Golden, CO (US)

(21) Appl. No.: 19/107,606

(22) PCT Filed: Aug. 29, 2023
(86) PCT No.: PCT/US2023/031442

§ 371 Date: Feb. 28, 2025

Related U.S. Application Data
(60) Provisional application No. 63/401,982, filed on Aug. 29, 2022.

(51) Int. Cl. B09B 3/38; B09B 3/32; B09B 3/35; B09B 3/40; B09B 3/60; B09B 101/25; C10L 5/46

(52) U.S. Cl. CPC B09B 3/38; B09B 3/32; B09B 3/35; B09B 3/40; B09B 3/60; C10L 5/46; B09B 2101/25; C10L 2200/0469; C10L 2270/04; C10L 2290/24; C10L 2290/26; C10L 2290/28; C10L 2290/30

(57) ABSTRACT

A process for preparing conversion-ready feedstock (CRF) is described. The process can be used to convert municipal solid waste (MSW) into CRF for the preparation of biochemicals, biofuels, biopower, and bioproducts. An exemplary process involves extruding MSW to provide a mechanically homogenized material that can be further processed prior to, during, or after extrusion (or another mechanical homogenization treatment) to provide the CRF. Also described am a system for preparing CRF from MSW and exemplary CRF made from converted MSW.

### CROSS REFERENCE TO RELATED APPLICATIONS

This application is a national stage filing of PCT International Application No. PCIYUS2023/031442, tiled Aug. 29, 2023, which claims benefit of and priority to U.S. Provisional Patent Application Ser. No. 63/401,982, tiled Aug. 29, 2022, the disclosure of each of which is incorporated herein by reference in its entirety.

### GOVERNMENT INTEREST

This invention was made with government support under DE-EE0009669 awarded by the U.S. Department of Energy and under Contract No. DE-AC36-080028308 between the United States Department of Energy and Alliance for Sustainable Energy, LLC, the Manager and Operator of the National Renewable Energy Laboratory. The government has certain rights in the invention.

### TECHNICAL FIELD

The presently disclosed subject matter relates, in some embodiments, to processes of converting waste, as well as systems related to the processes. More particularly, disclosed herein, in some embodiments are a process and system for converting municipal solid waste (MSW) into cost-effective and sustainable conversion-ready feedstocks for the production of biochemicals, biofuels, biopower, and bioproducts, including integrated biorefineries.

### BACKGROUND

Directing waste, such as municipal solid waste (MSW), to landfills results in a significant loss of material that could be transformed into cost-effective and sustainable conversion-ready feedstocks (CRF) for the production of biofuels, biopower, and bioproducts, including integrated biorefineries. The use of waste material to prepare CRF also has significant potential to reduce greenhouse gas (GHG) emissions. An estimated 292 million tons of MSW was generated in 2018 and over 5% was landfilled, resulting in 98 million metric tons of GHG emissions (CO₂e) from the biodegradable components of MSW (e.g., paper and food waste), making it the third largest source of anthropogenic methane emissions in the US.

Therefore, there is an ongoing need to develop MSW processing technologies to enhance the use of MSW as a source material in preparing CRF for the production of biofuels, biopower, and bioproducts for energy security and combating GHG emissions.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, the presently disclosed subject matter provides a process for preparing a conversion-ready feedstock (CRF) from a municipal solid waste (MSW), the process comprising: (a) mechanically homogenizing a MSW; and (b) performing one or more additional processing steps prior to, during, and/or after step (a), thereby preparing the CRF.

In some embodiments, step (b) comprises removing a hazardous material and/or a noncombustible material from the MSW prior to step (a). In some embodiments, step (b) comprises sorting a mixed MSW into one or more MSW fractions prior to step (a). In some embodiments, stop (b) comprises shredding the MSW prior to step (a). In some embodiments, step (b) comprises exposing the MSW to a liquid and/or a gas prior to or during step (a), optionally wherein the liquid and/or gas is steam and/or water.

In some embodiments, step (b) comprises treating the MSW to a treatment selected from the group comprising a decontamination treatment, a steam treatment, a washing treatment, a screening treatment, a homogenization treatment, a thermal treatment, a thermomechanical treatment, a thermochemical treatment, an AFEX treatment, an anhydrous ammonia treatment, a biological treatment, a biochemical treatment, a bio-thermochemical treatment, a biocatalyst treatment, a chemical catalyst treatment, a photocatalyst treatment, a sonication treatment, a microwave treatment, a photonic treatment, a pyrolysis treatment, a plasma treatment, an electron-beam treatment, an alkali treatment, a dilute acid pretreatment, a subcritical treatment, a supercritical CO₂ treatment, a deep eutectic mixture treatment, an enzymatic hydrolysis treatment and any combination thereof, optionally wherein the treatment is performed during step (a).

In some embodiments, step (b) comprises blending MSW with a non-MSW, optionally wherein the blending is performed after step (a) and comprises blending a mechanically homogenized MSW with a non-MSW. In some embodiments, step (b) comprises performing enzymatic hydrolysis on a mechanically homogenized MSW after step (a).

In some embodiments, step (a) comprises a method selected from the group comprising extruding, beating, refining, and milling, optionally wherein step (a) comprises extruding.

In some embodiments, the presently disclosed subject matter provides a system for preparing a conversion-ready feedstock (CRF), the system comprising: (i) a municipal solid waste (MSW); and (ii) a mechanical homogenization stage configured to receive the MSW from a source and mechanically homogenize the MSW to provide a preprocessed MSW fraction. In some embodiments, the system further comprises one or more processing stages configured to process the MSW.

In some embodiments, the mechanical homogenization stage comprises an extruder, optionally wherein the system further comprises a feeding stage configured to collect the MSW in a feed hopper and supply the MSW to the extruder. In some embodiments, the mechanical homogenization stage comprises a extruder, wherein the extruder comprises a die plate configured to extrude a homogenized MSW in a desired particle size, optionally wherein the die plate is configured at the end of a screw auger configured to move the MSW through the extruding stage. In some embodiments, the mechanical homogenization stage comprises an extruder comprising a screw auger configured to move the MSW through the extruder, optionally wherein the screw auger is powered and turned by a gear box and a drive mechanism. In some embodiments, the mechanical homogenization stage comprises a compression zone configured to compress the MSW, optionally wherein the compression zone is present in an extruder configured to extrude a homogenized MSW from a die plate in a desired particle size.

In some embodiments, the mechanical homogenization stage or the one or more processing stages comprise one or more treatment zones, optionally wherein the one or more treatment zones are selected from the group comprising a steam treatment zone, wherein the steam treatment zone is configured to provide steam to the MSW as it moves through the extruder; an alkaline treatment zone, wherein the alkaline treatment zone is configured to enhance enzymatic hydrolysis of the MSW; and an ammonia fiber expansion (AFEX) treatment zone, wherein the AFEX treatment zone is configured to enhance an enzymatic hydrolysis of the MSW.

In some embodiments, the mechanical homogenization stage comprises an extruder comprising a heating/cooling jacketed barrel configured to surround the MSW as it moves through the extruder.

In some embodiments, the one or more processing stages comprise a stage configured to screen, wash, and/or dry a MSW. In some embodiments, the one or more processing stages comprise a stage configured to blend a mechanically homogenized MSW provided from a mechanical homogenization stage with a non-MSW. In some embodiments, the one or more processing stages comprise a stage configured to perform an enzymatic hydrolysis reaction.

In some embodiments, the presently disclosed subject matter provides a conversion-ready feedstock (CRF) wherein the conversion-ready feed stock comprises a fully processed MSW. In some embodiments, the fully processed MSW is a biofuel, optionally wherein the biofuel is selected from the group comprising a sustainable aviation fuel (SAF), ethanol and a syngas, optionally wherein the syngas comprises a hydrogen fuel. In some embodiments, the fully processed MSW is a packaging, optionally a molded packaging or a flexible packaging. IN some embodiments, the fully processed MSW is a fermentable sugar.

Accordingly, it is an object of the presently disclosed subject matter to provide a process for preparing a CRF from a MSW, to a related system, and to the CRF itself. An object of the presently disclosed subject matter having been stated hereinabove, and which is achieved in whole or in part by the presently disclosed subject matter, other objects will become evident as the description proceeds herein below

### BRIEF DESCRIPTION OF THE DRAWINGS

FIG. 1 is a block flow diagram showing a process for converting carbohydrates in municipal solid waste (MSW) to sustainable aviation fuel (SAF) via a furans upgrading pathway and for converting plastics in MSW to syngas.

FIG. 2 is a schematic diagram showing a series of representative municipal solid waste (MSW) paper sample subcategories for homogenization.

FIG. 3 is a schematic diagram showing an extruder for use in a process for decontamination, homogenization, blending and pelletization of municipal solid waste (MSW).

FIG. 4 is a series of representative photographic images showing different exemplary materials in paper fractions of municipal solid waste (MSW), i.e., napkins, paper cups, pizza boxes, packages, mail and leaflets, and office paper. To the left-side is a representative photographic image of a mixed paper fraction of non-recyclable municipal solid waste (NMSW).

FIGS. 5A-5C are a series of representative photographic images showing municipal solid waste (MSW) paper fractions aster (FIG. 5A) sorting, (FIG. 5B) shredding, and (Figure SC) homogenization via extrusion treatment.

FIG. 6 is a graph showing particle size (volume percent (%) versus particle size in microns) of conversion-ready feedstock (CRF) prepared from various municipal solid waste (MSW) subfractions (mixed paper (dashed line); high-lignin paper (solid line with diamonds); high-ash paper (solid line with circles); high-cellulose, unwashed paper (dashed line with squares); and high-cellulose, washed paper (solid line with triangles) after extrusion treatment.

FIG. 7 is a graph showing enzymatic hydrolysis conversion (conversion percent (%) versus time (in hours)) of extruded paper subfractions of municipal solid waste (MSW). Conversion is shown for a high-cellulose washed subfraction (diamonds), a high-ash subtraction (triangles), a mixed paper subfraction (circles), a high-cellulose unwashed subtraction (symbol filled squares), and a high-lignin subtraction (symbol-less squares).

FIGS. 8A-8C are a series of representative photographic images showing a municipal solid waste (MSW) food fraction (FIG. 8A) after sorting, (FIG. 88) after extrusion, and (FIG. 8C) collected as a homogenized conversion ready feedstock (CRF).

FIG. 9 is a series of representative photographic images showing municipal solid waste (MSW) after 1) collection 2) sorting of plastics, 3) sorting of the plastic fraction to provide a polyethylene terephthalate (PET) subfraction 4) shredding/cutting of the PET subfraction. 5) extrusion treatment of the shredded PET subfraction, and 6) a collected homogenized conversion ready feedstock (CRF) from the PET subtraction.

FIGS. 10-10C are a series of representative photographic images showing a municipal solid waste (MSW) textile fraction (FIG. 10A) after sorting, (FIG. 10B) after extrusion, and (FIG. 10C) collected as a homogenized conversion ready feedstock (CRF).

FIG. 11 is a series of representative photographic images showing an exemplary screw auger suitable for use in a method of the presently disclosed subject matter.

FIG. 12 is a series of representative photographic images showing exemplary screw auger, blade, and feed hopper configurations for use in a method of the presently disclosed subject matter.

FIG. 13 is a series of representative photographic images showing an exemplary process and corresponding apparatus for preparing hand sheets for use in preparing a packaging from municipal solid waste (MSW) paper fractions.

FIG. 14 is a representative photographic image showing packaging hand sheets prepared from different municipal solid waste (MSW) paper subtractions, i.e., high-ash paper (upper left), high-lignin paper (upper right), mixed weight paper (lower left), and high-cellulose paper (lower right).

FIGS. 15A and 15B are schematic diagrams showing (FIG. 15A) a flow chart for an exemplary process for preparing a conversion-ready feedstock (CRF) from a municipal solid waste (MSW) including optional pre-extrusion processing, extruding, and post-extrusion processing steps and (FIG. 15B) additional details of an exemplary optional pre-extrusion processing of the process shown in FIG. 15A.

FIGS. 16A-16C are schematic diagrams showing (FIG. 16A) exemplary system 1600 for converting municipal solid waste (MSW) into a conversion-ready feedstock (CRF) of the presently disclosed subject matter including MSW 1601; optional pre-mechanical homogenization (e.g., pre-extrusion) processing stage 1610; feeding stage 1619, mechanical homogenization (e.g., extrusion) stage 1620; and processing stage 1630; (FIG. 16B) optional sub-stages of optional pre-mechanical homogenization (e.g., pre-extrusion) treatment stage 1610 shown in system 1600 of FIG. 16A; and (FIG. 16C) details of exemplary reeding stage 1619 and of exemplary mechanical homogenization stage 1620. The exemplary mechanical homogenization stage represents an extrusion-based stage further comprising exemplary processing stages.

### DETAILED DESCRIPTION

In some embodiments, the presently disclosed subject matter provides a process and system for preparing highly homogenized MSW that can be used tier bioproducts developments such as packaging, molded products, agriproducts, and construction materials. An exemplary system can include, for instance, components for: (a) collection of MSW from various public outlets, (b) separation of paper, plastics, textiles, and t xxi waste and removal of recyclables such as aluminum, iron, and glass. (c) separation of a paper fraction separated in (b) into high-cellulose content, high-lignin content, and high-ash content subfractions, (d) shredding of various subfractions of paper into small pieces (e.g., 1 to 5 inches), followed by sequential steaming to decontaminate and soften various subfractions. (e) extrusion processing to homogenize and concentrate into highly dense fractions, (f) drying of the homogenized fractions and (g) additional conversion processes for providing biochemicals, biofuels, biopower, and/or bioproducts.

The presently disclosed subject matter will now be described more fully. The presently disclosed subject matter can, however, be embodied in different forms and should not be construed as limited to the embodiments set firth herein below and in the accompanying Examples. Rather, these embodiments are provided so that this disclosure will be thorough and complete, and will fully convey the scope of the embodiments to those skilled in the art.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

Following long-standing patent law convention, the terms “a,” “an;” and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising,” which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim. As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising.” “consisting of,” and “consisting essentially of.” where one of these three terms is used herein, the presently disclosed and claimed subject matter can include the use of either of the other two terms. Unless otherwise indicated, all numbers expressing quantities of time, temperature, size, weight, concentration, volume, strength, speed, length, width, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value is meant to encompass variations of in one example ±25%, in one example ±20%, in one example ±10%, in another example ±5%, in another example ±1%, and in still another example ±0.1% from the specified amount, as such variations are appropriate to perform the presently disclosed methods and employ the presently disclosed systems.

Numerical ranges recited herein by endpoints include all numbers and fractions subsumed within that range (e.g., 1 to 5 includes 1, 1.5, 2, 2.75, 3.3.90, 4, 4.24, and 5). Similarly, numerical ranges recited herein by endpoints include subranges subsumed within that range (e.g., 1 to 5 includes 1-1.5, 1.5-2-2-2.75, 2.75-3, 3-3.94, 3.90-4, 4-4.24, 4.24-5,2-5, 3-5, 1-4, and 2-4).

The terms “non-recycled municipal solid waste.” “non-recyclable municipal solid waste.” “municipal solid waste” “municipal solid waste fraction,” “NMSW” and “MSW” are used herein interchangeably. The terms refer to paper, plastics, rubber, textiles, food, wood waste and/or other biomasses, metals, glasses that are found in, for example, landfills, household trash, office sites, restaurants and industrial sites.

The term “non-municipal solid waste.” “non-municipal solid waste fraction” and “non-MSW fraction” are used herein interchangeably. The term refers to materials not originating in MSW. The terms refer in some embodiments to grain starch. In some embodiments, the terms refer to carbohydrate-containing waste materials wherein the carbohydrate containing waste materials are selected from the group consisting of agro-residues, forest resides, sawdust, crops biomass such as rice straw, wheat straw, hemp hurds, sugarcane waste, perennial grasses, Miscanthus, algae, organic wet waste, industrial waste (such as sludge from pulp and paper mills), waste treatment plants, biocomposites, and any combination thereof. In some embodiments, the grain starch comprises corn, wheat, sorghum, other crops and any combination thereof.

The term “conversion-ready feedstock” and “CRF” are used herein interchangeably. The terms refer, in some embodiments, to a homogenized MSW of a uniform particle size, optionally with high carbohydrate and calorific value. In some embodiments, the homogenized CRF comprises a mechanically homogenized MSW. In some embodiments, the homogenized CRF comprises a mechanically and chemically homogenized MSW. In some embodiments, the homogenized CRF comprises a mechanically and physically homogenized MSW, in some embodiments, the homogenized CRF comprises a mechanically, physically, and chemically homogenized MSW.

The term “biomass” as used herein refers to any organic material that can be used as a conversion-ready feedstock for biofuel, biopower, and bioproducts. For example, in some embodiments, the term “biomass” refers to lignocellulosic plant-derived material. In some embodiments, the term “biomass” as used herein, refers to biomass materials segregated from other waste materials such as, but not limited to, food, paper, wood waste or yard trimmings/clippings segregated from MSW.

The term “lignocellulosic feedstock,” as used herein, refers in some embodiments to plants or byproducts from conversion, including trees, crops, forest residues, sawdust and agricultural residues.

In some embodiments, the term “lignocellulosic” refers to a composition comprising lignin and/or cellulose. In some embodiments, the term “lignocellulosic refers to a material comprising both lignin and cellulose. In some embodiments, lignocellulosic material can comprise hemicellulose, a polysaccharide which can comprise saccharide monomers other than glucose. Typically, lignocellulosic materials comprise between about 30-45 weight % cellulose, about 20-35 weight % hemicellulose; and about 3-35 weight % lignin.

“Lignin” is a polyphenolic material comprised of phenyl propane units linked by ether and carbon-carbon bonds. Lignins can be highly branched and can also be crosslinked. Lignins can have significant structural variation that depends, at least in part, on the plant source involved.

The term “cellulose” refers to a polysaccharide of β-glucose (i.e., β-1,4-glucan) comprising β-(1-4) glycosidic bonds. The term “cellulosic” refers to a composition comprising cellulose.

The term “hemicellulose” can refer polysaccharides comprising mainly sugars or combinations of sugars other than glucose (e.g., xylose). Thus, xylan (polymerized xylose) and mannan (polymerized mannose) are exemplary hemicelluloses. Hemicellulose can be highly branched, hemicellulose can be chemically bonded to lignin and can further be randomly acetylated, which can reduce enzymatic hydrolysis of the glycosidic bonds in hemicellulose.

The term “washing” and “washing stage” as used herein refer to the cleaning of MSW, a preprocessed MSW fraction or CRF, to remove unwanted debris or contaminants. In some embodiments, the washing stage is configured to wash with hot water. In some embodiments, the washing stage is configured to wash with a surfactant. In some embodiments, the washing stage is configured to wash with a defoamer, in some embodiments, the washing stage is configured to wash with a carbon dioxide acidification.

The terms “homogenization” and “homogenize” as used herein refer in some embodiments to the treatment (e.g., physical, mechanical, chemical or other treatment) by which a composition or structure or mixture of substances is made uniform in size and/or composition and/or other characteristic(s).

The terms “ammonia fiber expansion” and “AFEX” as used herein refer to a thermochemical pretreatment technology that can convert lignocellulosic biomass into a highly digestible conversion-ready feedstock

The terms “treatment” and “boated” as used herein refers to methods or processes that modify the physical, thermal, compositional, mechanical and/or chemical properties of a substance and/or of a modified substance.

The term “thermochemical processing” as used herein refers to the use of heat to promote chemical transformations of the MSW.

The term “ash” and “ash content” as used herein, refer to the inorganic content that would be left after ignition or complete oxidation of a biomass. In some embodiments, the ash content of the sample may comprise various residues from chemicals used in its manufacture, metallic matter from piping and machinery, mineral matter in the pulp from which the paper was made, and filling, coating, pigmenting and/or other added materials (see Tappi T 211 method). The amount and composition of the ash is a function of the presence or absence of any of these materials or others singly or in combination. In some embodiments, ash is determined by combustion of MSW at 525° C. in muffle furnace. In some embodiments, the ash is removed from the preprocessed MSW fraction.

The term “fully processed” as used herein, refers to when the MSW is converted into a desired end product.

The term “calorific value” refers to the energy contained in an MSW determined by measuring the heat produced by the complete combustion of a specified quantity of it.

As would be apparent to one of ordinary skill in the art upon a review of the instant disclosure, when referring to a particular characteristic (e.g., size, composition, concentration, viscosity, and the like) the term “uniform” can include 100% uniform but is not limited to 100% uniform. The term “uniform” can also include variations from a given value, such as a range around and/or based on the given value, and still be viewed as “uniform.” Thus, as used herein, the term “uniform,” when referring to a characteristic, is meant to encompass variations of in one example ±25%, in one example ±20%, in one example ±10%, in another example ±5%, in another example ±1%, and in still another example ±0.1° within, with respect to, and/or related to the specified characteristic (such as but not limited to size, composition, concentration, viscosity, and the like), as such variations are appropriate to perform the presently disclosed methods and/or employ the presently disclosed systems.

### II Processes and Systems for Preparing CRF

The presently disclosed subject matter provides, in one aspect, a process for preparing a conversion-ready feedstock (CRF). In some embodiments, the presently disclosed subject matter provides a process for preparing a CRF from MSW. Thus, for example, the process can be used to convert a heterogenous and/or bulky MSW composition into a denser, more homogenous composition, e.g., that is suitable for further conversion to a variety of products, e.g., biochemicals, biofuels, biopower, etc. In some embodiments, the process can include fully converting the MSW into a CRF that comprises a “fully processed MSW”, i.e., a desired end product or end products, such as a biochemical, a biofuel, biopower, or another bioproduct. Thus, in some embodiments, the presently disclosed subject matter relates to recycling MSW to provide a feedstock for a new product or products or for recycling the MSW into the new product or products themselves.

In some embodiments, the presently disclosed process comprises mechanically homogenizing the MSW or a fraction thereof (e.g., a fraction of MSW obtained by sorting MSW based on chemical composition). Mechanical homogenization can include any suitable mechanical homogenization process. The type of mechanical homogenization process used can, in some embodiments, depend upon the composition of the MSW and/or the desired properties of the CRF being produced. Mechanical homogenization can include, but is not limited to, extruding, beating (e.g., using a valley beater), refining, and milling (e.g., grinding). In some embodiments, the presently disclosed process comprises extruding MSW or a fraction thereof.

The mechanical homogenization (e.g., extrusion) can provide a denser and/or more homogenous material. In some embodiments, the denser and/or more homogeneous (e.g., extruded) material can be more readily or efficiently stored and/or transported for processing to provide the CRF for year-round availability in a factory. In some embodiments, the denser and/or more homogeneous (e.g., extruded) material can more readily or efficiently undergo processing (e.g., enzymatic hydrolysis, pyrolysis, etc.) to provide a high yield CRF. Thus, in some embodiments, the mechanically homogenized (e.g., extruded) material can be referred to as a “preprocessed MSW fraction.”

In some embodiments, the method further comprises performing one or more additional processing steps (i.e., in addition to the mechanical homogenization, such as the extruding). The one or more additional processing steps can be performed prior to, during (i.e., simultaneously with), and/or after the MSW is mechanically homogenized. Thus, in some embodiments, the presently disclosed subject matter provides a process for preparing a CRF from a MSW, wherein the process comprises: (a) mechanically homogenizing a MSW (e.g., extruding a MSW or a fraction thereof); and (b) performing one or more additional processing steps prior to, during, and/or after step (a), thereby preparing the CRF. Step (a) can comprise or consist of any suitable technique. In some embodiments, step (a) comprises or consists of one of the group including, but not limited to, extruding, beating, refining, and milling. In some embodiments, step (a) comprises or consists of extruding.

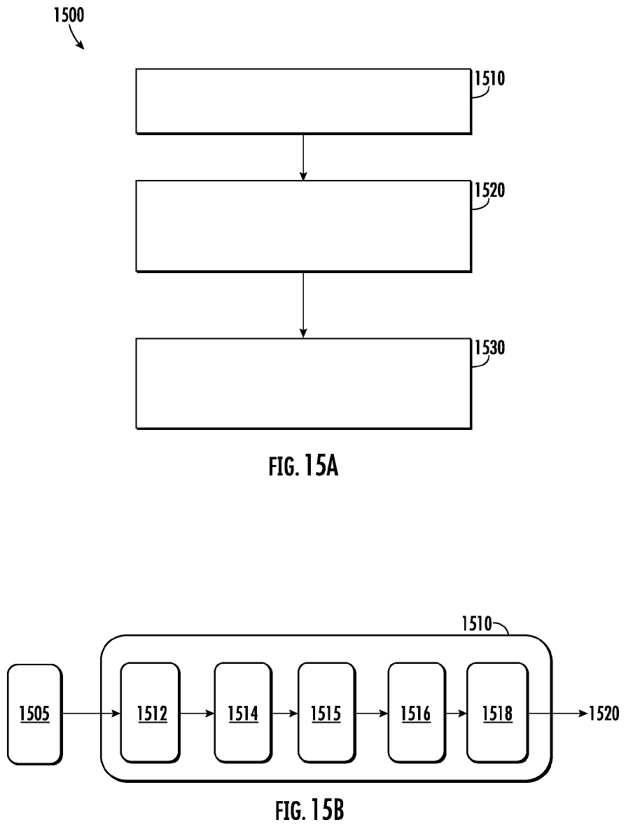

Thus, in some embodiments, the presently disclosed process comprises: (a) extruding a MSW to obtain a preprocessed MSW fraction, and (b) performing one or more additional processing step on the preprocessed MSW, in some embodiments, the process alternatively or additionally includes processing a MSW prior to the extruding. In some embodiments, the extruding and one or more additional processing steps take place simultaneously. FIG. 15A shows exemplary extrusion-based process 1500 for preparing a CRF from a MSW, which comprises optional pre-extrusion processing step 1510 (e.g., to produce pretreated MSW), extruding step 1520 to produce a mechanically homogenized MSW (also referred to herein as “an extruded MSW fraction”); and optional post-extrusion processing step 1530 to convert the mechanically homogenized MSW into a CRF.

In some embodiments, prior to extruding (or prior to another mechanical homogenization step) or prior to an optional pre-extrusion (or pie-mechanical homogenization) processing step, the MSW is collected from a waste site. The waste site can be any site that produces and/or collects waste, e.g., non-recycled municipal solid waste (NMSW). In some embodiments, the waste site is any waste site that produces and/or collects waste materials that comprise combustible waste (e.g., combustible biomass-derived waste materials), such as, but not limited to, food waste, textile waste, paper or other lignocellulosic waste, rubber waste, and/or plastic waste. In some embodiments, the waste site comprises a residential collection facility. In some embodiments, the waste site comprises a residential dumpster. In some embodiments, the waste site comprises a trash bin. In some embodiments, the waste site comprises a restaurant (e.g., a trash bin and/or dumpster associated with and/or collecting waste from a restaurant). In some embodiments, the waste site comprises a landfill, in some embodiments, the waste site comprises a recycling center. In some embodiments, the waste site comprises a corporate office.

In some embodiments, the waste site comprises an industrial site. In some embodiments, the waste site comprises a community center. In some embodiments, the waste site comprises a transfer station. In some embodiments, the waste site comprises a material recycling facility or material recovery facility (MRF), also known as clean MRF or dirty MRF. The clean MRF process single-stream recycling materials whereas dirty MRF process mixed waste and extract recyclable and other materials from garbage or trash.

In some embodiments, the method comprises performing a processing step prior to mechanically homogenizing (e.g., prior to extruding) the MSW. For instance, the process comprises performing, prior to the mechanical homogenizing of step (a), one or more of: removing a hazardous material from the MSW, removing a noncombustible material from the MSW, separating the MSW into a MSW fraction or sub-fraction (e.g., based on composition/MSW type), sizing the MSW or a fraction or subtraction thereof (e.g., to reduce the size of the MSW components or provide uniform particles of MSW), and preconditioning the MSW or a fraction or subtraction thereof (e.g., by treating it with a liquid and/or gas, optionally under pressure and/or at an elevated temperature).

For example, in some embodiments, prior to step (a) (e.g., prior to extruding), a hazardous material (e.g., a hazardous waste) is separated from the MSW (e.g., the MSW as obtained from the waste site). In some embodiments, the hazardous material comprises a bio-hazardous waste. In some embodiments, the hazardous material comprises contaminated personal protective equipment (PPE). In some embodiments, the hazardous material comprises electronic components. In some embodiments, the hazardous material comprises chlorinated and/or fluorinated components, including fluorochemicals, also known as forever chemicals. In some embodiments, the hazardous material comprises large gaseous, liquid, or solid waste containers. In some embodiments, the hazardous material comprises animal waste, such as cat litter.

In some embodiments, the process comprises removing a noncombustible material from the MSW prior to step (a). In some embodiments, the noncombustible material comprises metal. In some embodiments, the metal comprises aluminum. In some embodiments, the metal comprises ferrous metals. In some embodiments, the noncombustible material comprises glass.

In some embodiments, the process comprises separating the MSW into an MSW fraction prior to step (a), wherein the MSW fraction comprises similar types of materials. In some embodiments, the MSW is separated into an MSW subfraction (e.g., by further separating a MSW fraction, such that each MSW subfraction comprises a single type of material and/or a mixture of materials representing a subgrouping of materials present in a MSW fraction. e.g., that share one or more property).

More particularly, in some embodiments, prior to step (a), the process comprises or consists of detection and sorting (which can also be referred to more simply as “sorting”) the MSW into one or more fractions and/or subtractions. Detection and sorting can be performed, for example, using standard industrial practices, such as those used in materials recycling/recovery and/or at waste disposal facilities. These practices can include visual detection and manual hand sorting and/or conveyor-based optical detection and pneumatic sorting. In some embodiments, the MSW object detection and sorting can comprise the use of an integrated artificial intelligence (AI). In some embodiments, the integrated AI comprises an enabled camera. In some embodiments, the enabled camera comprises multispectral and/or hyperspectral imaging cameras. In some embodiments, the integrated AI comprises a pneumatic system that sorts the MSW into different fractions of MSW to be extruded and processed. In some embodiments, the integrated AI comprises a robotic arm to sort the MSW into different fractions of MSW.

In some embodiments, the detecting and sorting comprises using a conveyor-based system. In some embodiments, the conveyor-based system comprises the enabled camera. The enabled camera can be selected from the group comprising a visual camera, a multispectral camera, a hyperspectral imaging camera and combinations thereof. In some embodiments, the conveyor-based system comprises one or more sensors. For instance, each of the one or more sensors can be selected from a moisture sensor, a temperature sensor, and a gas sensor. In some embodiments, the one or more sensors are configured to make informed decisions on the overall quality of the MSW or fractions or subfractions thereof. In some embodiments, the conveyor-based system uses an operator wherein the operator wears integrated AI augmented reality/visual reality (AR/VR) glasses to sort the MSW into different fractions of MSW.

In some embodiments, the MSW is sorted into one or more of a MSW plastic fraction, a MSW food fraction, a MSW paper fraction, a MSW textile fraction, and a MSW yard waste fraction. In some embodiments, the sorted MSW fraction comprises “other” waste, e.g., material from MSW that cannot be categorized into one of the previously described MSW fractions. For example, the sorted MSW fraction can comprise composite materials (e.g., fiberglass), hazardous materials, or other materials not otherwise encompassed or fully encompassed by another MSW fraction.

In some embodiments, the sorting comprises sorting the MSW to provide a MSW plastic fraction comprising, consisting essentially of, or consisting of plastic waste. The plastic materials of the MSW plastic fraction can include any plastic waste, such as, but not limited to, waste comprising polyesters (e.g., polyethylene terephthalate (PET or PETE), polyolefins (e.g., polyethylene (PE), high-density polyethylene (HDPE), low-density polyethylene (LDPE), and polypropylene (PP)), polyvinyl chloride (PVC), polystyrenes (PS), acrylic or methacrylic polymers (e.g., polymethyl methacrylate (PMMA) and any combination thereof (e.g., an acrylonitrile-butadiene-styrene (ABS) block copolymer). In some embodiments, the MSW plastic fraction comprises, consists essentially of, or consists of one or more of PET, HDPE, PP, and PS. In some embodiments, the MSW plastic fraction comprises, consists essentially of, or consists of one or more of an acrylic polymer (e.g., polyacrylonitrile (PAN), a methacrylic polymer (e.g., PMMA), HDPF, LDPE, PC, PE, PP, PET, PVC, and ABS. In some embodiments, the MSW plastic fraction comprises, consists essentially of, or consists of one or more category of plastic waste based on intended/former use or a mechanical or physical property, such as single-use plastics, plastic bags or films, rigid plastics, disposables, utensils, and others. In some embodiments, the MSW plastic fraction can be further sorted into MSW plastic subfractions based on chemical composition (e.g., polyolefins can be separated from non-polyolefin plastic waste or can be separated by particular type of polyolefin, such as into separate PP, PE, HOPE, and LDPE subfractions), density, melting point, or another mechanical or physical property.

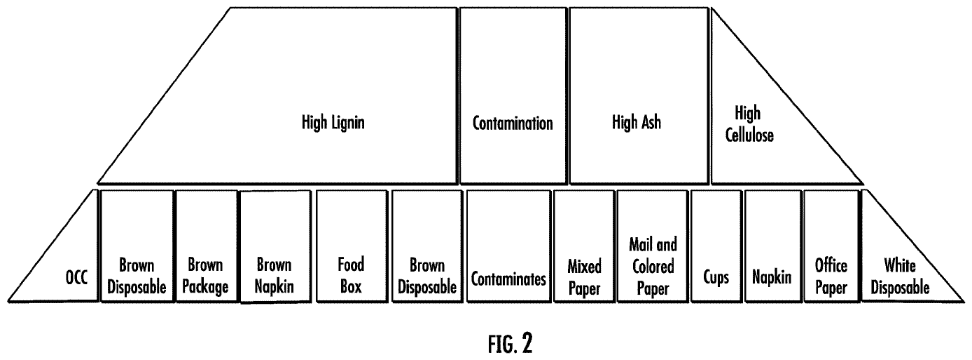

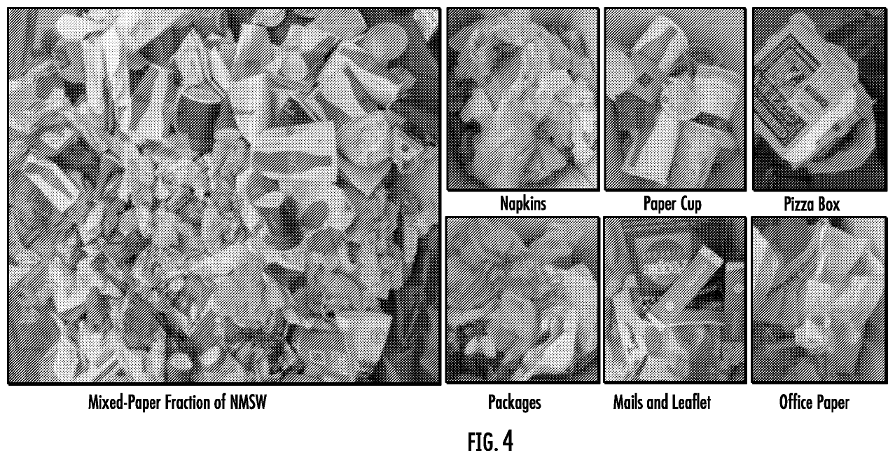

In some embodiments, the sorting provides a MSW paper fraction comprising, consisting essentially of, or consisting of paper waste. Paper waste can include, but is not limited to, office paper, napkins, old corrugated containers (OCC), secondary boxes, envelopes, newspapers, glossy papers, brown packages, disposables, cups, tetra packs, mixed paper, and others. As shown in FIG. 2, the MSW paper fraction (i.e., the MSW mixed paper fraction) can further be sorted into MSW paper subfractions, such as a high-lignin paper fraction (e.g., containing M.C, brown disposable papers, brown packages, brown napkins, food boxes, etc.), a MSW high-ash paper fraction (e.g., mail, colored paper, and cups), and a MSW high-cellulose paper fraction (e.g., napkins, office paper, and other white disposable papers). FIG. 4 also shows (at left) a MSW mixed paper fraction and various MSW paper subfractions based on paper use-type.

In some embodiments, the sorting provides a MSW food fraction comprising, consisting essentially of, or consisting of food waste. In some embodiments, a food of the food group is selected from the group comprising fruits, vegetables, meats, and others. In some embodiments, the sorting comprises further sorting the MSW food group to separate and/or remove denser materials, such as chicken bones.

In some embodiments, the sorting provides a MSW textile fraction comprising, consisting essentially of, or consisting of textile waste. The textile waste can include materials comprising cotton fibers, synthetic fibers (e.g., acrylic fibers), mixed cotton and synthetic fibers, natural fibers (e.g., silk), wood-based fibers and others.

In some embodiments, the sorting provides a MSW yard waste fraction comprising, consisting essentially of, or consisting of yard waste. The yard waste of the MSW yard waste fraction can comprise leaves, grass, twigs, and other plant-based waste.

In some embodiments, the one or more processing steps comprise sizing the MSW prior to step (a) (e.g., reducing the size of the MSW or making the MSW of a more uniform size). Thus, in some embodiments, the process comprises a sizing step such as, but not limited to, shredding, cutting, chopping, grinding and/or pulverizing. In some embodiments, the sizing comprises shredding the MSW or a MSW fraction prior to step (a). The shredding can be performed manually or mechanically. In some embodiments, the shredding comprises shredding the MSW or MSW fraction into uniform particles.

In some embodiments, the process comprises preconditioning the MSW (or a fraction or subfraction thereof) to provide a preconditioned MSW prior to step (a). In some embodiments, the preconditioning comprises washing, wetting (e.g., using water or steam), soaking, and/or heating the MSW or a fraction or subfraction thereof. In some embodiments, the MSW is soaked in water. In some embodiments, prior to step (a), the MSW is decontaminated via atmospheric plasma.

FIG. 15B shows a flow diagram of an exemplary optional pre-extrusion processing step 1510 (including several individual optional processing steps) that can be performed according to the presently disclosed and claimed process prior to extruding step 1520 of FIG. 15A. FIG. 158 also shows step 1:405, representing obtaining MSW from a waste site. In pre-extrusion processing step 1510, the as-obtained MSW can undergo step 1512 for removing hazardous material, step 1514 for removing non-combustible material, step 1515 for sorting the MSW into fractions or subfractions based on waste type/composition, step 1516 for sizing the MSW, and step 1518 for preconditioning the MSW. In a process of the presently disclosed and claimed subject matter, the optional pre-extrusion processing can be performed wherein steps 1512, 1514, 1515, 1516, and 1518 are performed in the order indicated in FIG. 158 or, alternatively, in any other suitable order. In some embodiments, pre-extrusion processing step 1510 comprises a subset of steps 1512, 1514, 1515, 1516, and 1518. For example, in some embodiments, any one, two, three, or four of steps 1512, 1514, 1515, 1516, and 1518 are performed in step 1510, in some embodiments, all five of steps 1512, 1514, 1515, 1516, and 1518 are performed prior to extruding step 1520.

The number, order, and identity of the steps 1512, 1514, 1515, 1516, and 1518 performed in pre-extrusion processing step 1510 can depend upon the composition of MSW obtained from the waste site (or otherwise provided for conversion to a CRF according to the presently disclosed process) and/or the type of CRF desired and/or the efficiency of the process desired. In some embodiments, when the sorting comprises providing a MSW plastic fraction, the pre-extrusion processing can further comprise conditioning (e.g., washing) and shredding the MSW plastic fraction prior to the extruding. In some embodiments, when the sorting comprises providing a MSW paper fraction, the pre-extrusion processing can further comprise shredding and preconditioning the MSW paper fraction with steam. In some embodiments, when the sorting comprises providing a MSW paper fraction, the pre-extrusion processing can further comprise preconditioning the MSW paper fraction with water and shredding the MSW paper fraction.

In some embodiments, when step (a) comprises extruding, the presently disclosed process comprises feeding the MSW or a fraction or subfraction thereof into an extrusion hopper (e.g., after the pre-extrusion preprocessing). In some embodiments, the extrusion hopper is attached to an extruder.

In some embodiments, when step (a) comprises extruding, the extruding comprises extruding the MSW using screw auger. In some embodiments, the extruding comprises extruding the MSW through a single-screw or multi-screw extruder (e.g., a twin-screw extruder). In some embodiments, the extruding comprises extruding the MSW through a die plate configured to extrude a desired particle size. In some embodiments, the extruding comprises heating the MSW during the extrusion at one or more different temperatures.

In some embodiments, the extruding further comprises exposing the MSW to a liquid and/or gas (e.g., simultaneously extruding the MSW and exposing it to a liquid and/or gas). In some embodiments, the liquid and/or gas is selected from the group consisting of steam, water, and any combination thereof. In some embodiments, the liquid and/or gas (e.g., the steam) is provided at a temperature of about 100° C. to about 350° C. Thus, in some embodiments, the extruding is performed using an extruder configured to supply steam to the MSW during the extrusion process. In some embodiments, the extruder is configured to extrude a moist or moistened MSW. The moistened MSW can be moistened by the steam in the extruder or premoistened prior to being placed in the extruder.

In some embodiments, the one or more additional processing steps (i.e., step (b) comprises treating the MSW to a treatment, such as, but not limited to, a decontamination treatment, a steam treatment, a washing treatment, a screening treatment, a homogenization treatment, a thermal treatment, a thermomechanical treatment, a thermochemical treatment, an AFEX treatment, an anhydrous ammonia treatment, a biological treatment, a biochemical treatment, a bio-thermochemical treatment, a biocatalyst treatment, a chemical catalyst treatment, a photocatalyst treatment, a sonication treatment, a microwave treatment, a photonic treatment, a pyrolysis treatment, a plasma treatment, an electron-beam treatment, an alkali treatment, a dilute acid treatment, a subcritical treatment, a supercritical CO₂ treatment, a deep eutectic mixture, an enzymatic hydrolysis treatment, and combinations thereof. In some embodiments, the treatment is performed during step (a) (e.g., during extrusion). In some embodiments, the treatment is selected from a group comprising, but not limited to, a hydrothermal treatment, an anhydrous ammonia treatment, a biocatalyst treatment, a chemical catalyst treatment, a photocatalyst treatment, a sonication treatment, a microwave treatment, a photonic treatment, a pyrolysis treatment, a plasma treatment, an electron-beam treatment, an alkali treatment, a dilute acid pretreatment, a subcritical water treatment, a supercritical CO₂ treatment, deep eutectic mixture treatment, and any combination thereof. In some embodiments, the treatment comprises the AFEX treatment. In some embodiments, the treatment is an alkaline treatment. In some embodiments, the treatment comprises a hydrothermal treatment.

Thus, in some embodiments, when step (a) comprises extruding, the extruding is performed in an extruder configured to apply a treatment (such as one of the treatments described above) to the MSW. Thus, in some embodiments, the MSW is treated in the extruder or in a portion of the extruder.

In some embodiments, the mechanical homogenization (e.g., the extruding) comprises homogenizing (e.g., extruding) MSW to obtain one or more desirable characteristics. For example, in some embodiments, the desirable MSW characteristics can be based on a property selected from the group including, but not limited to, particle size, surface area, viscosity, rheology, moisture, carbohydrate level, lignin content, calorific value, heating value, high heating value (HHV), low heating value (LHV), sulfur content, pH, ash content and any combination thereof.

In some embodiments, step (b) comprises sorting the MSW after step (a) into different fractions, such as organics and plastics for integrated biorefineries to produce biofuels, biopower and bioproducts or any combination thereof. In some embodiments, the processing comprises blending the preprocessed MSW fraction with a non-MSW fraction (e.g., to enhance one or more properties of the MSW to make it a more suitable CRF source material, such as to enhance the calorific value of the preprocessed MSW). In some embodiments, the blending is performed after step (a). Thus, in some embodiments, the process comprises blending a mechanically homogenized MSW (e.g., an extruded MSW) with a non-MSW. In some embodiments, the non-MSW fraction is a material that contains a carbohydrate. In some embodiments, the non-MSW fraction is a grain starch. In some embodiments, the non-MSW fraction is a carbohydrate-containing waste material that enhances calorific value.

In some embodiments, the MSW is converted to a CRF that can be used as a feedstock for another product. In some embodiments, the process comprises or further comprises converting the CRF to a final product. In some embodiments, the final product is sustainable aviation fuel (SAP).

In some embodiments, step (b) comprises a treatment (e.g., after step (a)) that converts a chemical compound in the MSW (e.g., in a mechanically homogenized MSW) into another chemical intermediate and/or compound by breaking one or more chemical bonds and/or by making one or more chemical bonds. In some embodiments, step (b) comprises hydrolysis (e.g., enzymatic hydrolysis), pyrolysis, fermentation, electrolysis, and/or gasification.

In some embodiments, step (b) comprises performing enzymatic hydrolysis a mechanically homogenized (e.g., extruded) MSW. Thus, in some embodiments, the mechanically homogenized (e.g., extruded) MSW is contacted with one or more enzymes under conditions suitable for enzymatic activity. Any suitable enzyme or mixture of enzymes can be used. In some embodiments, the enzyme comprises or consists of cellulase. In some embodiments, the enzyme of the enzymatic hydrolysis comprises or consists of amylase. In some embodiments, the enzyme is selected from the group comprising, but not limited to, cellobiohydrolase, endo-1,4-β-glucanase, hemicellulases, xylanases, endo-β-xylanases and β-xylosidase, lignin peroxidase, glucanase, mannanase, xyloglucan hydrolase and pectinase, α-1-arabinofuranosidase, α-galactosidase and α-glucuronidase, acetyl xylan esterase, feruloyl esterase, glucuronoyl esterase and any combination thereof.

In some embodiments, step (b) comprises performing a thermochemical process (e.g., after step (a)), such as, pyrolysis of a mechanically homogenized (e.g., extruded) MSW at a high temperature in an anaerobic environment. In some embodiments, step (b) comprises performing a biochemical process, such as, but not limited to, fermenting a mechanically homogenized (e.g., extruded) MSW fraction in the presence of biocatalysts such as enzymes and/or microorganisms. In some embodiments, step (b) comprises performing a thermochemical process, such as performing gasification of mechanically homogenized (e.g., extruded) MSW fraction at high temperatures under controlled air, oxygen, steam and/or other conditions. In some embodiments, the thermochemical process comprises a gasification of mechanically homogenized (e.g., extruded) MSW fraction with a plasma microwave furnace or plasma torch. In some embodiments, the thermochemical process comprises a gasification of a mechanically homogenized (e.g., extruded) MSW plastic fraction with a plasma torch to produce industrial gas mixtures, referred as “synthesis gas” or syngas. In some embodiment %, step (b) comprises performing a combination of thermochemical and biochemical process comprises first a gasification of mechanically homogenized (e.g., extruded) MSW fraction into syngas followed by fermentation of syngas to produce biofuels. In some embodiments, the “syngas” is a mixture of mainly carbon monoxide and hydrogen. In some embodiments, the syngas is burned in a conventional boiler to produce steam electricity. In some embodiments, step (b) comprises producing electricity via direct combustion of mechanically homogenized (e.g., extruded) MSW in a boiler to produce high-pressure steam that flows over turbine blades. The rotating turbine blades can drive a generator producing electricity. In some embodiments, the mechanically homogenized (e.g., extruded) MSW can serve as a substitute for at least a portion of coal in the power generation plant furnace in a process called co-firing.

In some embodiments, step (b) comprises converting mechanically homogenized (e.g., extruded) MSW into syngas. In some embodiments, step (b) further comprises using the syngas as a C1-building block for the production of commodities and fine chemicals. In some embodiments, the carbon monoxide in syngas produced by processing a mechanically homogenized (e.g., extruded) MSW can yield another molecule of hydrogen through the use of the water-gas shift reaction (CO+H₂O→CO₂+H₂). In some embodiments, hydrogen is obtained after removing the carbon dioxide (CO₂). In some embodiments, the hydrogen from the syngas prepared by processing a mechanically homogenized (e.g., extruded) MSW is used for the synthesis of ammonia, methanol, hydrogen peroxide, and/or other fine chemicals. In some embodiments, the hydrogen from the syngas provided as a fuel for powering automobiles. In some embodiments, the hydrogen from syngas is used for the hydrogenation of oils and fats, and/or the manufacturing of drugs and fine chemicals. In some embodiments, the removed carbon dioxide (CO₂) from hydrogen is converted to C1-C3 building blocks using an electrolysis process. In some embodiments, the syngas is referred as “medium-BTU gas” having a heating value of 300 to 400 Btu per cubic foot. In some embodiments, the mechanically homogenized (e.g., extruded) MSW is gasified to yield syngas which is then catalytically converted via the Fischer-Tropsch Process (F-T), also referred to “indirect liquification,” to produce gasoline and diesel fuel. In some embodiments, the mechanically homogenized MSW is treated to remove sulfur to enhance catalytic conversion via Fischer-Tropsch Process (F-T) process. In some embodiments, syngas produced from the mechanically homogenized MSW is used as a fuel to produce methanol, which is then used as a chemical feedstock for the natural gas production. In some embodiments, the methanol is used as a principal chemical, a chemical precursor or clean fuel. The terms “principal chemical” or “chemical precursor” or “chemical feedstock” as used herein in some embodiments refer to the use of syngas as a C1-building block to produce gaseous and liquid fuels, commodities chemicals, and/or fine chemicals.

In some embodiments, step (b) comprises performing a thermochemical process comprising an aqueous ammonia treatment. In some embodiments, the aqueous or liquid ammonia treatment of lignocellulosic biomass alters the structure of cellulose I to the more reactive cellulose III_{I} (CIII) allomorph, which is more amenable towards enzymatic hydrolysis to yield monomeric sugars. In some embodiments, treatment with liquid ammonia results in the cleavage of ester-linked ferulates and coumarate linkages that exist in grass cell walls via ammonolysis. In some embodiments, the aqueous ammonia treatment is performed after step (a).

The presently disclosed subject matter provides, in some embodiments, an integrated municipal solid waste (MSW) processing system for providing conversion-ready feedstocks (CRF), e.g., useful for the conversion of MSW to biochemicals, biofuels, biopower, and other bioproducts. Thus, for example, the system can be configured to convert heterogeneous, bulky, and highly variable MSW into highly dense and homogenous CRFs suitable or conversion to biochemicals, biofuels, biopower, and/or other bioproducts. In some embodiments, the system comprises a mechanical homogenization stage (e.g., an extrusion-based stage), to convert a bulky and/or heterogenous MSW or a fraction thereof to provide a more homogenous and/or dense composition, in some embodiments, the system can comprise a biorefinery or can be integrated with a biorefinery. In some embodiments, the MSW processing system provides for conversion of a MSW to a one or more CRFs comprising one or more fully processed MSWs, such as one or more biochemicals, biofuels or other bioproducts.

Accordingly, in some embodiments, the presently disclosed subject matter provides a system for preparing a CRF. In some embodiments, the system comprises (i) a MSW, (ii) a mechanical homogenization stage configured to receive the MSW from a source (e.g., a waste site, a pretreatment processing stage or a feeding stage) and mechanically homogenize the MSW to provide a preprocessed MSW fraction. In some embodiments, the system further comprises one or more processing stages configured to press the SMW.

In some embodiments, the system comprises a processing stage configured to process or “pretreat” the MSW prior to mechanical homogenization in the mechanical homogenization stage. The pre-extrusion processing stage can comprise one or more components configured to sort, separate, shred, decontaminate (e.g., remove hazardous material), condition, and/or remove non-combustible materials.

For example, in some embodiments, the system comprises a processing stage having the capability to sort, shred, and/or decontaminate the MSW. In some embodiments, the processing stage comprises one or more components configured for use in manual sorting (e.g., bins and/or a conveyor belt). In some embodiments, the system includes a conveyor-based sorting component (e.g., a conveyor-based optical sorting component). In some embodiments, the one or mom components configured for use in sorting comprise an integrated AI configured to sort MSW into different fractions of MSW to be further processed. In some embodiments, the integrated AI comprises an enabled camera. In some embodiments, the enabled camera comprises a hyperspectral imaging camera. In some embodiments, the integrated AI comprises a pneumatic system that sorts the MSW into different fractions of MSW to be processed. In some embodiments, the integrated AI comprises a robotic arm to sort the MSW into different categories of MSW to be processed.

In some embodiments, the system includes a conveyor-based system (e.g., as part of a processing stage). In some embodiments, the conveyor-based system comprises the enabled camera selected from the group comprising a visual camera, a multispectral camera, a hyperspectral imaging camera and any combination thereof. In some embodiments, the conveyor-based system comprises sensors wherein the sensors are selected from the group consisting of a moisture sensor, a temperature sensor, a gas sensor, and any combination thereof. In some embodiments, the sensors are configured to make informed decisions on the overall quality of the material and the conversion pathways. In some embodiments, the conveyor-based system comprises an operator wherein the operator wears an integrated artificial intelligence augmented reality/visual reality (AR/VR) glass to sort the MSW into different categories of MSW to be further processed.

In some embodiments, the system comprises one or more components configured to shred MSW (e.g., as part of a processing stage). In some embodiments, the system comprises one or more industrial shredder mechanical devices to reduce the size of the MSW for the extrusion system.

In some embodiments, the system comprises one or more components configured to decontaminate and/or condition MSW (e.g., as part of a processing stage). In some embodiments, the system comprises one or more components for plasma and steam injection. For example, the one or more components can comprise a plasma system, e.g., which can include cold atmospheric plasmas or plasma torches. In some embodiments, the system can comprise screening, cleaning, and washing systems, such as those that are readily available in recycling facilities.

The mechanical homogenization stage can comprise components suitable for any desired mechanical homogenization technique. In some embodiments, the mechanical homogenization stage comprises an extruder, a beater (e.g., a valley beater), a refiner, or a mill. In some embodiments, the mechanical homogenization stage comprises an extruder (e.g., consists or comprises an “extrusion stage”). In some embodiments, the mechanical homogenization stage comprises an extruder, the system can further comprise a feeding stage configured to collect the MSW in a feed hopper and supply the MSW to the extruder. In some embodiments, a feeding stage depth is constant. In some embodiments, the feeding stage is configured to convey the MSW on a conveyor and supply the MSW to the extruder. In some embodiments, the feeding stage is configured to control the feed rate of MSW to the extruder. In some embodiments, the feeding stage is configured to flood feed the extruder, wherein the hopper is filled, and extruder controls the uptake of the material.

In some embodiments, the feeding stage is configured to precondition the MSW in the feed hopper with a thermal processing component and supply the MSW to the extruder. In some embodiments, the thermal component is selected from the group comprising atmospheric plasma, hot air, hot water and any combination thereof.

The extruder can comprise a single-screw extruder, a twin-screw extruder, or another multi-screw extruder. In some embodiments, the extruder is a single screw extruder or twin-screw extruder. In some embodiments, the extruder (e.g., the single or twin-screw extruder) can comprise a one or more temperature control zones, for providing control of the temperature in part or all of the extruder. In some embodiments, the extruder (e.g., the single or twin-screw extruder) comprises a plurality of temperature control zones, thereby providing for extrusion over different temperatures. In some embodiments, the extruder or extrusion stage can further include water and steam injection zones to sullen the MSW before and/or during the extrusion process.

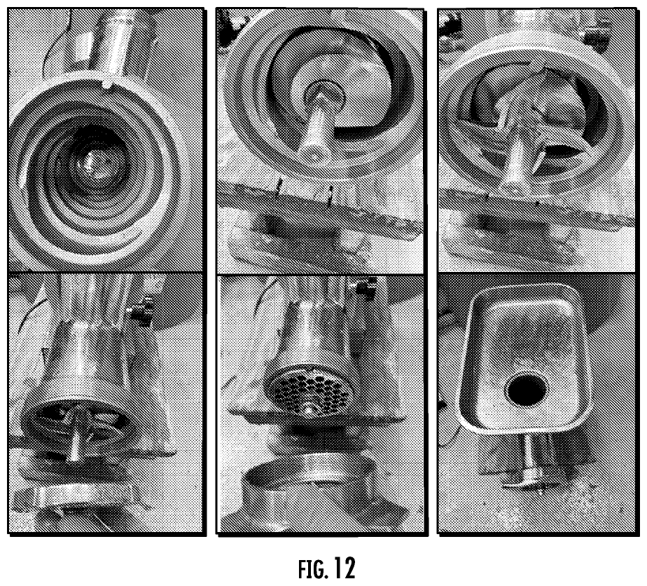

For example, in some embodiments, the extruder comprises a screw auger for moving the MSW through the extruder. See FIG. 11. In some embodiments, the screw auger comprises a single screw auger. In some embodiments, the screw auger comprises a double screw auger. In some embodiments, the screw auger comprises twin screws. In some embodiments, the screw auger comprises multi screws. In some embodiments, the multi screws may rotate in opposite directions. In some embodiments, the multi screws may rotate in the same directions. In some embodiments the flights of the screws intermesh. In some embodiments, the flights of the screws partially intermesh. In some embodiments, the flights of the screws may not intermesh.

In some embodiments, the screw auger is mechanically turned. In some embodiments, the screw auger is powered and turned by a gear box and a drive mechanism. In some embodiments the screw auger is a hydraulic cylinder. In some embodiments, the hydraulic cylinder is an air pressure hydraulic cylinder. In some embodiments, the hydraulic cylinder is a liquid pressure cylinder. In some embodiments, the screw auger is configured a blade for cutting MSW prior to extrusion. See FIG. 12.

In some embodiments, the extruder comprises a screw auger in a tube or a barrel. In some embodiments, the extruding stage comprises U-shaped barrel. In some embodiments, the extruding stage comprises a barrel with holes. In some embodiments, the screw is configured with large groove flutes in a pair with a corresponding inlet and outlet with a barrier ridge between them to shear and homogenize MSW to provide a uniform particle size. In some embodiments, the screw is tapered to provide the screw with a larger diameter at the feed end.

In some embodiments, the extruder is configured with a barrier screw with an extra flight to form two parallel channels with different clearance, where small particles can pass over while large particles experience additional frictional force providing uniform particle size and viscosity.

In some embodiments, the mechanical homogenization stage (e.g., the extruder in a mechanical homogenization stage) comprises a die plate. In some embodiments, the die plate is configured at an end of the screw auger. In some embodiments, the die plate is configured at an end of the extruder. In some embodiments, the die plate is configured to extrude the preprocessed MSW in a desired particle size. In some embodiments, the die plate is configured to have a plurality of holes. In some embodiments, the holes of the die plate can comprise any diameter necessary to achieve the desired particle size.

In some embodiments, the mechanical homogenization stage comprises a compression zone. In some embodiments, the compression zone is configured to compress the MSW. In some embodiments, the compression zone is present in an extruder configured to move the MSW to a die plate configured to extrude homogenized MSW from the die plate in a desired particle size In some embodiments, the compression zone depth gets narrow to build the pressure and remove air between the MSW particles. In some embodiments, the mechanical homogenization stage comprises an extruder and the extruder is configured to reduce the pressure significantly to apply vacuum through a hole in the barrel (vent) to remove air, moisture, extractives, proteins, fats, volatiles, and the like. In some embodiments, the extruder is configured with vent hole to add liquid, solids, or gaseous materials. In some embodiments, the mechanical homogenization stage can include mixing and/or kneading sections. In some embodiments, the extruder of a mechanical homogenization stage comprises mixing and/or kneading sections or zones. In some embodiments, the extruder includes a cascade section, where the output from one screw may feed the second screw.

In some embodiments, the compression ratio of the extruder is greater than 1. In some embodiments, the compression ratio is less than 4. In some embodiments, the compression ratio is the ratio of the volume of the first flight to the last flight of the screw. In some embodiments, the compression zone moves the MSW to the die plate.

In some embodiments, the extruder is twin-screw mounted on splined shaft in a barrel. In some embodiments, the extruder has multi-barrel modules for heat treatments, chemical injections, filtration, screening, fractionations, hydrolysis, degassing, gas injections, liquid injections, solid separation, washing, bleaching and combination thereof. In some embodiments, the extruder can be used as a reactive polymer processing to change melt behavior and rheology.

In some embodiments, the extrusion process is a continuous process with a modular design. In some embodiments, the temperature of the extruder barrel ranges from room temperature to 400° C. In some embodiments, the temperature of the barrel ranges from room about room temperature to 300° C. In some embodiments, the temperature of the barrel is about 150° C. In some embodiments, the pressure of the barrel ranges from atmospheric pressure to 300 bar. In some embodiments, the pressure of the barrel ranges is 200 bars. In some embodiments, the extruder comprises a twin-screws extruder, and the screws co-rotate with a preset clearance. In some embodiments, the screw diameter is 1 inch. In some embodiments, the screw diameter is 10 inches. In some embodiments, the screw speed is 50 rpm. In some embodiments, the extruder can produce 10,000 lb. per hour preprocessed MSW. In some embodiments, the extruder can produce 1lb. per hour preprocessed MSW. In some embodiments, the extruder can produce 10,000 lb. per hour preprocessed MSW.

In some embodiments, the mechanical homogenization stage and/or the one or more processing stages comprise one or more treatment zones, such as, but not limited to a steam treatment zone, an alkaline treatment zone, and an ammonia fiber expansion (APEX) treatment zone.

In some embodiments the system (e.g., the mechanical homogenization stage and/or one of the one or more processing stages) comprises a steam treatment zone. In some embodiments, the steam treatment zone is configured to provide a steam to the MSW as it moves through the mechanical homogenization stage. For example, in some embodiments, the system comprises an extruder comprising an integrated steam treatment zone. In some embodiments, the steam is configured to decontaminate the MSW. In some embodiments, the steam is configured to sterilize the MSW.

In some embodiments, the system (e.g., the mechanical homogenization stage and/or one of the one or more processing stages) comprises an alkaline treatment zone. In some embodiments, the alkaline treatment zone is configured to enhance enzymatic hydrolysis of the preprocessed MSW. In some embodiments, the alkaline treatment zone is integrated in the mechanical homogenization stage (e.g. integrated in an extruder). In some embodiments, the extruding stage comprises a dilute acid treatment zone, wherein the dilute acid treatment zone is configured to enhance the fibrillation and enzymatic hydrolysis of the preprocessed MSW.

In some embodiments, the system (e.g., the mechanical homogenization stage and/or one of the one or more processing stages) comprises an AFEX treatment zone. In some embodiments, the AFEX treatment zone is configured to enhance enzymatic hydrolysis of the preprocessed MSW. In some embodiments, the AFEX treatment zone is incorporated in an extruder. In some embodiments, the extruding stage comprises a biological treatment zone, wherein the enzyme pretreatment zone is configured to reduce the particle size of the preprocessed MSW.

In some embodiments, the mechanical homogenization stage comprises an extruder comprising a heating/cooling jacketed barrel. In some embodiments, the heating/cooling jacketed barrel is configured to surround the MSW as it moves through the extruder or a portion thereof. In some embodiments, the heating/cooling jacketed barrel is configured to surround the extruder or a portion thereof. In some embodiments, the heating/cooling jacketed barrel facilitates thermomechanical preprocessing. In some embodiments, a screw tip temperature is configured to less than 180° C. to avoid degradation of certain MSW through the extruding stage. In some embodiments, the screw tip temperature is controlled by changing the frictional force through the clearance between the screw tip and the inner wall of the barrel.

In some embodiments, the one or more processing stages comprise a screening stage configured to screen preprocessed (e.g., mechanically homogenized) MSW. In some embodiments, the one or more processing stages comprise a washing stage configured to wash preprocessed MSW. In some embodiments, the one or more processing stages comprise a drying stage of the preprocessed MSW. In some embodiments, the one or more processing stages comprise a stage configured to blend the preprocessed MSW fraction with non-MSW fractions. In some embodiments, the one or more processing stages further comprise a formulation of preprocessed MSW organic faction and a non-MSW fraction. In some embodiments, the one or more processing stages further comprise a formulation of preprocessed MSW paper waste fraction and a non-MSW fraction. In some embodiments, the one or more processing stages further comprise a formulation of preprocessed MSW food waste fraction and a non-MSW fraction.

In some embodiments, the one or more processing stages comprise an enzymatic hydrolysis stage. In some embodiments, the enzymatic hydrolysis is performed by cellulase. In some embodiments, the enzymatic hydrolysis is performed by amylase.

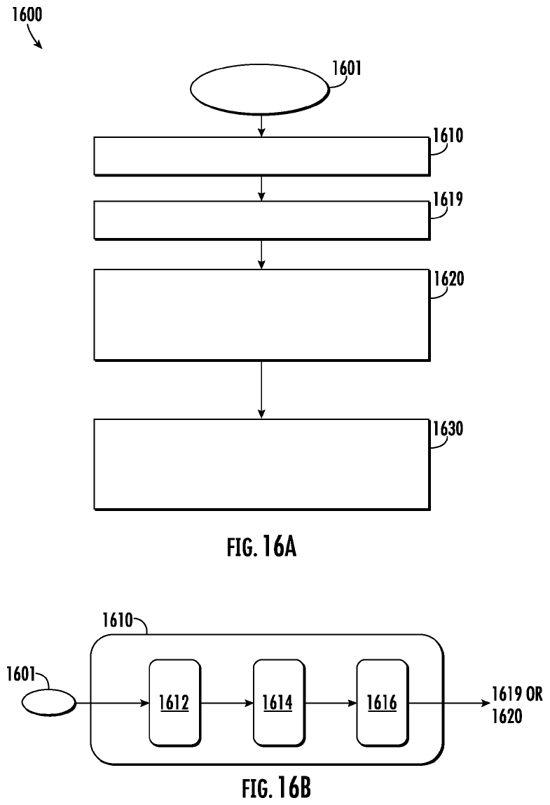

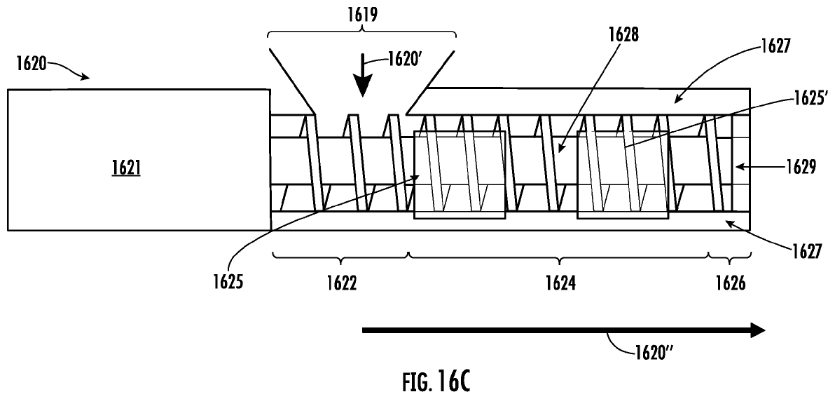

FIG. 16A shows exemplary system 1600 of the presently disclosed subject matter. System 1600 comprises MSW 1601 (e.g., obtained from a waste site) and an optional pre-mechanical homogenization processing stage 1610, which is configured to provide MSW from stage 1610 to optional feeding stage 1619. Alternatively, in a system without a pre-mechanical homogenization processing stage. MSW 1601 can be provided directly to optional feeding stage 1619 or directly to mechanical homogenization stage 1620. Optional feeding stage 1619 is configured to provide MSW to mechanical homogenization stage 1620, which can be for example, an extrusion stage, comprising an extruder. Mechanical homogenization stage 1620 is configured to provide preprocessed MSW to processing stage 1630.

FIG. 16B shows an exemplary pre-mechanical homogenization processing stage 1610 of a system of the presently disclosed subject matter. Exemplary processing stage 1610 includes a sorting stage 1612 configured to separate MSW 1601 into fractions and subfunctions. For example, sorting stage 1612 can include one or more of a conveyor belt, a camera, an integrated AI, a series of containers to hold sorted fractions or subfractions, and a robotic arm. In some embodiments, sorting stage 1612 can be configured to remove hazardous and/or non-combustible material from MSW 1601. Sorting stage 1612 is configured to provide material to shredding stage 1614. Shredding stage 1614 can comprise a shredder to shred one or more of the MSW fractions or subfractions from sorting stage 1612. Shredded MSW from shredding stage 1614 is then provided to conditioning/decontamination stage 1616, which can include, for example, a steam or plasma injection system to treat the shredded MSW. From conditioning/sorting stage 1616, MSW is then provided to feeding stage 1619 or mechanical homogenization stage 1620 of a system of the presently disclosed subject matter. The relative configuration of stages 1612, 1614, and 1616 in pre-mechanical homogenization processing stage 1610 can be changed depending upon the nature of the MSW and/or the properties desired in the preprocessed MSW fraction or corresponding CRF. In some embodiments, one or more stages 1612, 1614, or 1616 can be omitted or replaced by a stage configured to perform another processing step.

FIG. 16C shows an exemplary extrusion/extruder-based mechanical homogenization stage 1620 of a system such as shown in FIG. 16A. Exemplary stage 1620 comprises screw auger 1628, which is powered by gear box and drive mechanism 1621 to move MSW provided to stage 1620 via feeding stage 1619, e.g., a feed hopper. More particularly feeding stage 1619 is configured to feed MSW in direction 1620′ into feed zone 1622 of stage 1620. As screw auger 1628 moves the MSW through stage 1620 in the direction 1620″, the MSW moves through compression zone 1624 and reaches extrusion zone 1626, where it is extruded through die plate 1629. Screw auger 1628 is housed in heating/cooling jacketed barrel 1627, which further comprises treatment zones 1625 and 1625′. For example, in some embodiments, treatment zone 1625 can be a steam treatment zone configured to inject steam into the MSW as it moves through mechanical homogenization (e.g., extrusion) stage 1620. Treatment zone 1625′ can be an alkaline treatment zone configured to inject an alkaline composition or an AFEX treatment zone configured to inject liquid ammonia.

In some embodiments, the presently disclosed subject matter provides a CRF. In some embodiments, the CRF comprises a fully processed MSW. In some embodiments, the fully processed MSW comprises fermentable sugars. In some embodiments, the fully processed MSW comprises a sugar stream comprising 5- and 6-carbon sugars. In some embodiments, the fully processed MSW comprises 2,5-disubstituted furan derivatives, such as 2,5-dimethylfuran and 2,5-diformylfuran provided via a conversion process via a 5-(hydroxymethyl)furfural (HMF) platform chemical. In some embodiments, the fully processed MSW comprises furans and furan derivatives provided via a conversion process from a furfural platform. In some embodiments, the fully processed MSW comprises 5-(hydroxymethyl)furfural (HMF) and furfural platform molecules. In some embodiments, the fully processed MSW comprises a 2,5-Furandicarboxylic acid (FDCA). In some embodiments, the fully processed MSW comprises FDCA based polymers such as Polyethylene Furanoate (PEF).

In some embodiments, the fully processed MSW comprises a Glycerol platform chemical, such as Mannitol. In some embodiments, the fully processed MSW comprises a Sorbitol platform chemical, such as Sorbose. In some embodiments, the fully processed MSW comprises Levulinic acid platform chemicals, such as γ-Valerolactone and Adipic acid. In some embodiments, the fully processed MSW comprises Glutamic acid platform chemicals, such as Glutaminol, Norvoline and 1,5-Pananediol. In some embodiments, the fully processed MSW comprises Glucaric acid platform chemicals, such as a Gluconic acid, Glucoronic acid and Methylglucoside. In some embodiments, the fully processed MSW comprises Itaconic acid platform chemicals, such as laconic diamide and 3-Methyl tetrahydrofuran.

In some embodiments, the fully processed MSW comprises 1,4-Diacid platform chemicals such as Succinic, Fumaric, and Malic. In some embodiments, the fully processed MSW comprises 1,4-Diacid platform polymers such as 1,4-butanediol (1,4-BDO). In some embodiments, the fully processed MSW comprises Lactic acid platform chemicals such as Lactates and Pyruvic acid. In some embodiments, the fully processed MSW comprises ABE platform chemicals such as Acetone, Butanol and Ethanol.

In some embodiments, a fully processed MSW comprises a biofuel. In some embodiments, the biofuel is a sustainable aviation fuel. In some embodiments, the biofuel is ethanol. In some embodiments, the biofuel comprises diesel blend stock. In some embodiments, the fully processed MSW comprises a composition useful for providing biopower. In some embodiments, fully processed MSW comprises a syngas. In some embodiments, the syngas is used to produce electrical power. In some embodiments, the syngas comprises a hydrogen fuel. In some embodiments, the syngas is a hydrogen fuel. In some embodiments, the syngas is used as a hydrogen fuel, in some embodiments, a hydrogen fuel comprises syngas. In some embodiments, a hydrogen fuel is a syngas. In some embodiments, the syngas comprises a biofuel. In some embodiments, the syngas is a biofuel. In some embodiments, the syngas is used as a biofuel. In some embodiments, a biofuel comprises syngas. In some embodiments, a biofuel is a syngas. In some embodiments, the syngas comprises a chemical precursor. Biopower can also encompass heat or electricity generation from preprocessed MSW or chemical intermediates such as syngas from preprocessed MSW.

In some embodiments, the fully processed MSW is a packaging. In some embodiments, the packaging is a molded packaging. In some embodiments, the packaging comprises a Linerboard or corrugated medium. In some embodiments, the packaging is a flexible packaging. In some embodiments, the packaging is a single use item. In some embodiments, the packaging is a disposable packaging.

In some embodiments, the fully processed MSW comprises a bioplastic.

### EXAMPLES

The following examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1: The Process

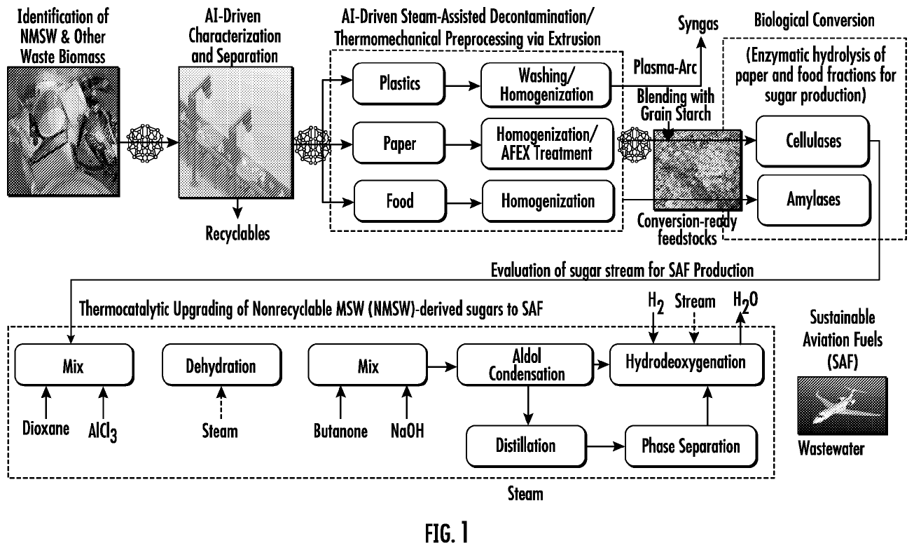

An exemplary process of the presently disclosed subject matter includes integrated Artificial Intelligence (AI)-driven sequential hydrothermal, mechanical and biochemical preprocessing of NMSW to selectively remove unwanted constituents upstream and enhance ultimate feedstock compositional, structural, and physical attributes to deliver conversion-ready feedstock (CRF) for the production of biofuels and bioproducts. The process steps synergistically couple (a) real-time characterization to feed-in physical and compositional attributes for appropriate preprocessing of NMSW and for blending grain starch and other waste feedstocks; (b) an AI-driven cascade preprocessing integrating: i) removal of hazardous and non-combustible recyclables such as aluminum, ferrous metals, and container glass, and ii) separation of various fractions such as paper, plastics, textiles, and food, followed by sequential hydrothermal, mechanical and biochemical preprocessing to decontaminate, homogenize, concentrate and blend the carbohydrate-rich fraction of NMSW with grain starch with targeted structural attributes for effective convertibility for selected conversion processes; (c) conversion of carbohydrate fractions (paper, food and textile streams) for CRF for biofuels such as SAF; (d) conversion of paper fractions to bioproducts such as molded packaging and composites; and (e) conversion of the plastic fraction to SAF or syngas via plasma torch treatments, providing for electricity, hydrogen, biohydrogen or chemical manufacturing from syngas. A block flow diagram for converting the carbohydrates in NMSW into SAF via furans upgrading pathway and plastics to syngas is shown in FIG. 1.

### Example 2: MSW Sampling and Sorting for Homogenization Via Extrusion

MSW was obtained through NC State's Waste Reduction and Recycling (WRR) facility, which collects large amounts of versatile waste generated from residential halls, mixed-used buildings, and academic buildings. A single-family residential hall, a single occupancy residential hall, a library (as an example of a mixed-use building), and an academic building were selected for sampling. A total MSW sample of 242 lb. was collected.

Sorting was performed according to the ASTM D 5231 standard for manual sorting of MSW waste. Briefly, the sorting was performed as follows: 1) Collected over 200 lbs. of NMSW sample in black trash bags and tied snugly, 2) Unloaded the sample on a clean tarp, 3) Removed hazardous materials, such as syringes, nails, sharp glasses, etc., 4) Sorted NMSW in major fractions: Papers, Plastics, Food, Textiles, Metals, Glasses, Yard Wastes/Wood Wastes, and Others.

After sorting, the weight of each fraction (as a percent of the total MSW sample) was measured and recorded. The moisture content of each fraction was also measured. More particularly, for each fraction, a minimum of 2 pounds of the fraction was selected randomly after initial sorting. Initial weight was recorded (Wi). Final weight (Wf) was recorded after drying the samples under the sun/atmospheric conditions when less than 0.2% weight change was observed after successive weighing of the samples after 24 hours.

The following equation was used to calculate moisture (%):

Moisture Content (%)=((WI−Wf))/Wi×100

This process was developed to allow the testing of large quantities of the samples without losing any volatiles, avoid order issues, and avoid fire hazardous (due to the heterogeneous nature of the MSW materials) due to high-temperature conditions under standard moisture measurement protocols.

The MSW composition from the NC State WRR facility illustrated a typical MSW waste composition profile in which food, paper and plastics were the three major waste items by weight. See Table 1 A, below. The MSW food fraction showed the highest moisture content followed by the MSW paper fraction. The MSW plastic fraction also showed some moisture, which, without being tied to any one theory, is believed to be due to contamination from food and beverages.

TABLE 1A
Weight Fraction and Moisture Content
of Non-recycled MSW (NMSW).

| | | | Food | | | | |
|---|---|---|---|---|---|---|---|
| MSW Fraction | Paper | Plastic | waste | Textiles | Metal | Glass | Others |
| Weight | 30 | 20 | 34.5 | 3.3 | 2.7 | 1.3 | 8.2 |
| Fraction (%), | | | | | | | |
| As Collected | | | | | | | |
| Moisture (%) | 18.9 | 3 | 56 | 15 | | | |

The sorted MSW fractions were further analyzed; for example, the paper is fraction was further categorized by visual inspection and relevant physical, chemical, thermal and compositional properties. See FIG. 2. To evaluate the role of contaminants such as lignin and ash and remove contaminants that could influence downstream processing of a mixed paper feedstock, the paper fraction was sorted into four subfractions: (1) a high-cellulose subfraction (comprising bleached paper materials as a control subfraction with low lignin/ash contaminants); (2) a high-lignin subfraction (comprising unbleached materials); (3) a high-ash subfraction (comprising coated or laminated paper materials, rich in ash and plastic contaminant), and (4) a contamination fraction (comprising mixed paper and contaminants from other fractions of waste such as food). To understand the impact of heterogeneity, a control composition was also prepared based on the weighted fractions of the individual paper subfractions, referred herein as “mixed paper”, “mixed weight fraction” or “paper-mix.” The paper-mix fraction represents the combined fraction of high-cellulose, high-lignin and high-ash content fractions.

Samples of subfractions (high-lignin, high-ash, high-cellulose) and the mixed paper composition were extruded using steam and/or water assisted extrusion. Selected samples were also pretreated prior to extrusion via washing and/or screening to remove contaminants. Select samples were also (1) shredded and exposed to steam before extrusion; (2) soaked (e.g., by keeping the sample in water to soften) and then shredded prior to extrusion, or (3) shredded and extruded without any prior steam or water treatment or soaked (e.g., by keeping the sample in water to soften). Alternatively, paper fractions were not mechanically homogenized via extrusion, but were instead soaked in water, mechanically homogenized using a valley beater, and washed using a screen to removed contaminants.

Table 1B, below, summarizes the samples described further in the remaining examples. With the exception of sample #6 and sample #7, all samples were obtained through the NC WRR facility. Sample #6 was obtained from a commercial source and sample #7 from recycle bins.

TABLE 1B
Sample information and Processing Steps for MSW Samples.

| Sample | | | | | | | Sample |
|---|---|---|---|---|---|---|---|
| Type | Processing Steps | | | | | Category | IDs |
| NMSW | Sorted | Steamed | Shredded | Extruded | NA | Paper | #1 Mixed |
| | | | | | | | Weight |
| | | | | | | | Fraction |
| NMSW | Sorted | Steamed | Shredded | Extruded | NA | | #2 High |
| | | | | | | | Lignin |
| NMSW | Sorted | Steamed | Shredded | Extruded | NA | | #3 High |
| | | | | | | | Ash |
| NMSW | Sorted | Steamed | Shredded | Extruded | NA | | #4 High |
| | | | | | | | Cellulose |
| NMSW | Sorted | Water | Shredded | Valley | Water | Paper | #5 High |
| | | Soaked | | Beating | Washed | | Cellulose |
| | | | | | | | Washed |
| Virgin | NA | NA | NA | NA | NA | pulp | #6 |
| Hardwood | | | | | | | Hardwood |
| Pulp | | | | | | | pulp |
| Recyclable | NA | NA | NA | NA | NA | Brown | #7 Old |
| | | | | | | Paper | Corrugated |
| | | | | | | | Containers |
| | | | | | | | (OCC) |
| NMSW | Sorted | NA | Shredded | Extruded | NA | Paper | #8 Mixed |
| | | | | | | | Weight |
| | | | | | | | Fraction |
| NMSW | Sorted | NA | Shredded | Extruded | NA | | #9 High |
| | | | | | | | Lignin |
| NMSW | Sorted | NA | Shredded | Extruded | NA | | #10 High |
| | | | | | | | Ash |
| NMSW | Sorted | NA | Shredded | Extruded | NA | | #11 High |
| | | | | | | | Cellulose |
| NMSW | Sorted | NA | Shredded | Extruded | NA | Food | #12 Food |
| | | | | | | Waste | Waste |
| NMSW | Sorted | Water | Shredded | Extruded | NA | Paper | #13 Mixed |
| | | Soaked | | | | | Weight |
| | | | | | | | Fraction |
| NMSW | Sorted | Water | Shredded | Extruded | NA | | #14 High |
| | | Soaked | | | | | Lignin |
| NMSW | Sorted | Water | Shredded | Extruded | NA | | #15 High |
| | | Soaked | | | | | Ash |
| NMSW | Sorted | Water | Shredded | Extruded | NA | | #16 High |
| | | Soaked | | | | | Cellulose |
| NMSW | Sorted | Water | Shredded | Extruded | NA | Textile | #17 |
| | | Soaked | | | | | Textile |
| NMSW | Sorted | Water | Shredded | Extruded | Water | Paper | #18 Mixed |
| | | Soaked | | | Washed | | Weight |
| | | | | | | | Fraction |
| NMSW | Sorted | Water | Shredded | Extruded | Water | | #19 High |
| | | Soaked | | | Washed | | Lignin |
| NMSW | Sorted | Water | Shredded | Extruded | Water | | #20 High |
| | | Soaked | | | Washed | | Ash |
| NMSW | Sorted | Water | Shredded | Extruded | Water | | #21 High |
| | | Soaked | | | Washed | | Cellulose |
| NMSW | Sorted | NA | Shredded | Extruded | NA | Plastic | #22 All |
| | | | | | | | plastic |
| | | | | | | | (mixed |
| | | | | | | | fraction) |
| NMSW | Sorted | NA | Shredded | Extruded | NA | | #23 PET |

### Example 3: Steam-Assisted Extrusion Pretreatment of NMSW to Decontaminate MSW and Overcome Recalcitrance and Physical Heterogeneity

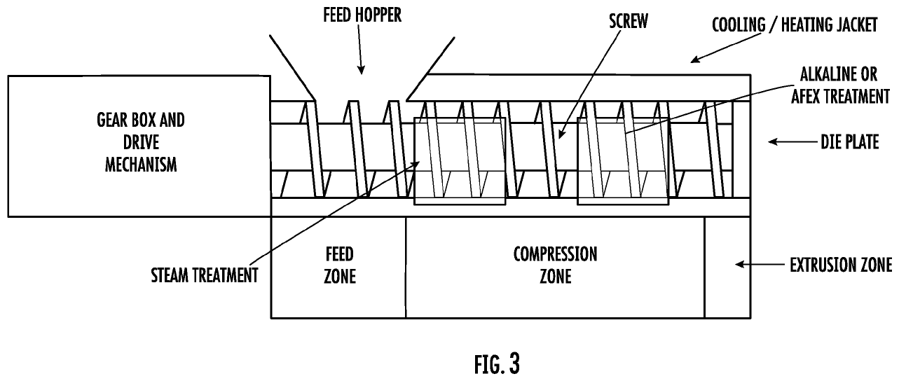

Since ~60-70% fraction of MSW is biodegradable, collection must be done rapidly. Other challenges related to the use of MSW include safety concerns due to potential presence of biohazardous material. Recalcitrance related to MSW can be reduced by treatment with steam, acid, or base at elevated temperatures. According to the presently disclosed subject matter, in some embodiments, a steam treatment is used for decontamination and for opening the pore structure of MSW material in combination with extrusion, used as a highly versatile and effective physical continuous homogenization treatment for MSW (e.g., for MSW used to prepare CRF). Compared to other MSW treatment methods, advantages of steam-assisted extrusion are: (1) no sugar degradation or inhibitory products generation; (2) low capital and operational costs, better process monitoring, and control of variables with AI; (3) high versatility and adaptability to various process modifications; and (4) continuous throughput/consistent uniform feed-output. An advanced preprocessing technology utilizing steam-assisted extrusion pretreatment is described below. See also FIG. 3.

In screw extruding, as the screw rotates, it transports bulk material forward while impacting physical, structural, mechanical, and chemical properties of the feed. The screw configuration provides control over the extent of mixing, shearing, flowability, transportation, extrudate properties, mechanical energy input, and residence time during extrusion, all of which can be utilized for process controls at an industrial scale. A series of representative images of a screw auger are shown in FIG. 11. As the MSW material is fed into the extruder, it is exposed to mixing, shearing, pressure, and temperature changes due to frictional heat to deconstruct the biomass structure in a short residence time. As it exits the extruder, a sudden pressure drop results in fibrillation and fiber breakdown, particle size reduction for higher surface area, accessibility, decrease in bulk density, and an increase in specific porosity. A series of representative images showing a screw auger, blade, and feed hopper configuration are provided in FIG. 12. Inputs, such as feed rate, steam injection, power, screw speed, etc. can be manipulated to get the desired outputs, such as pressure, temperature, viscosity (torque), particle size (visual camera), and pH.

Further, an extrusion pretreatment can be combined with chemical treatments to influence chemical properties. To evaluate contaminants in a MSW paper fraction, it was sorted into subfractions as described in Example 2, including: (I) a high-cellulose subfraction (e.g., bleached paper, as a control material with low lignin/ash contaminants); (2) a high-lignin subfraction (e.g., unbleached paper); and (3) a high-ash subfraction (e.g., coated paper). The high-cellulose/bleached paper subfraction can be homogenized using extrusion to overcome the heterogeneity in physical attributes of the bleached paper fraction. The steam pretreatment coupled with high shear tierces in the extruder can break fiber bundles and alter cell wall architecture, making them accessible to enzymatic hydrolysis. For the high-lignin/unbleached paper subfraction, extrusion pretreatment can be conducted with a mild alkaline treatment (e.g., 50-200 mM NaOH) at, for example 80° C.-100° C., for good final properties. AFEX (extractive-ammoniac) treatment can be used on unbleached paper, because it can convert and retain crystalline cellulose 1β (CI) to a highly digestible cellulose IIII (CIII) allomorph and extract up to ~45% of lignin. The bulk densities of the various paper subfractions (both “bulk density” (i.e., density of an “as collected”/prior to extrusion sample) and “homogenized density” (i.e., density after extrusion and pre-extrusion treatment comprising shredding and exposure to steam) are shown in Table 2, below. Homogenized density was increased in all samples via extrusion.

TABLE 2
Comparison of Pre-Processed MSW Paper
Fraction and Bulk Sample Density.

| | | Homogenized Density |
|---|---|---|
| | | (gm/L) |
| | Bulk Density | (shredded and exposed to |
| Sample type | (gm/L) | steam before extrusion) |
| High cellulose | 43.3 | 127.6 |
| High Ash | 53.2 | 147.6 |
| High Lignin | 46.6 | 124 |
| Mixed Weight Fraction | 49.2 | 152.4 |

### Example 4: Blending of Paper-Mix after Extrusion Pretreatment to Overcome Chemical Heterogeneity and Improve Feedstock Quality

Quality improvement of conversion ready feedstock (CRF) can be useful in providing CRFs with chemical, physical, and biological characteristics that better meet conversion specifications. As described hereinabove, a steam-assisted extrusion pretreatment can result in preprocessed MSW for use in preparing CRF with significant improvements in physical and biological characteristics (highly dense, high surface area, homogenous and accessible to enzymatic hydrolysis). In some embodiments, to overcome compositional variability, a homogenized CRF paper fraction can be blended with grain starch, using a compositionally consistent industrial waste stream, to enhance CRF qualities such as carbohydrate content, calorific value, sugar content, and others.

### Example 5: Extrusion Pretreatment of MSW Paper Fraction

FIG. 4 shows different subfractions of an MSW paper fraction. To evaluate the role of contaminants and calcitrant influencing downstream processing, the MSW paper fraction was sorted into various sub fractions according to the scheme shown in FIG. 2. The composition of these sorted subtractions was performed using NREL laboratory analytical procedures (LAPs) (NREL/TP-510-42618) shown in Table 3, below. Generally, the samples were shredded and exposed to steam prior to extrusion. The carbohydrate content increased by >32% for a mildly washed (e.g., washed using room temperature water on a wire-mesh screen under atmospheric conditions) high-cellulose paper fraction compared to the mixed fraction. The high-cellulose paper fraction was homogenized via a valley beater, followed by mild washing using an open screen. Samples prepared from hardwood pulp and old corrugated containers (OCC) were used as controls. The total carbohydrate fractions of different paper-rich MSW subfractions after extrusion pretreatment were similar to those of the pretreated biomass samples.

TABLE 3
Composition of various paper subfractions (shredded and exposed
to steam before extrusion) of non-recycled MSW (NMSW) and
two control feedstocks, i.e., hardwood pulp and OCC. (Data
provided as weight percentages (%) of noted components).

| | | | | Total | |
|---|---|---|---|---|---|
| | | | | Carbo- | Acid |
| Composition | Glucan | Xylan | Arabinan | hydrate | Insoluble |
| # 1 Mixed | 50.4 | 16.4 | 1.2 | 68.1 (±2.8) | 10.6 |
| Weight | | | | | |
| Fraction | | | | | |
| # 2High Lignin | 58.2 | 19.2 | 2.1 | 79.5 (±1.3) | 10.5 |
| # 3 High Ash | 52.0 | 15.8 | 1.0 | 68.8 (±1.1) | 18.7 |
| # 4 High | 55.2 | 17.6 | 0.5 | 73.4 (±1.3) | 1.9 |
| Cellulose | | | | | |
| # 5 High | 65.7 | 20.2 | 1.3 | 87.2 (±1.2) | 5.0 |
| Cellulose | | | | | |
| washed* | | | | | |
| # 6 Hardwood | 69.8 | 23.0 | 0.0 | 92.8 (±0.3) | 1.2 |
| Pulp | | | | | |
| # 7 Old | 53.2 | 17.8 | 1.8 | 72.8 (±0.1) | 15.2 |
| Corrugated | | | | | |
| Containers | | | | | |
| (OCC) | | | | | |

*Cellulose paper fraction soaked in water, mechanically homogenized using a valley beater, and washed using a screen to remove contaminants

### Example 6: Evaluation of the Quality of Homogenized Various MSW Paper Fractions

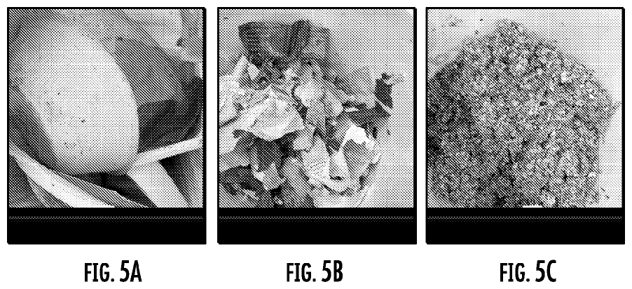

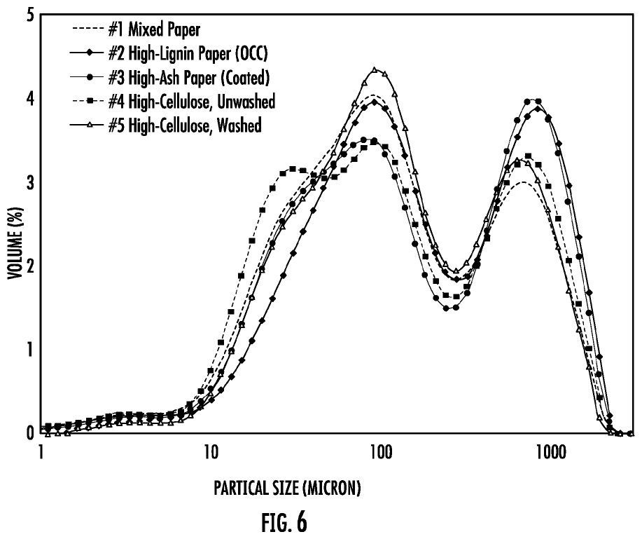

To evaluate extrusion pretreatment, induced homogenization particle size analysis was performed on various MSW paper subfractions. FIG. 4 shows images of a paper-mix fraction of different types of paper fractions, such as cups, plates, unbleached, coated, etc. FIGS. 5A and 5B show a paper-mix fraction before (FIG. 5A) and after (Figure SB) manual shredding to provide uniform particle sixes to feed into the extruder. FIG. 5C shows the extruded paper fraction, which provides a highly dense and homogenous conversion-ready feedstock for bioproducts, biochemicals, and biofuels conversions. The particle size determination of aqueous suspensions of homogenized paper fractions was obtained by laser diffraction (using a particle size analyzer sold under the tradename MASTERSIZER™ 2000 (Malvern Instruments Limited, Malvern, United Kingdom)), which measures the dimensions of a particle and reports a particle size as the diameter of an equal volume sphere. The results show particle size distribution ranging from 0.1-5 mm for all fractions, suggesting extrusion was very effective in reducing particle size for the majority of material to <5 mm. See FIG. 6.

Ultimate analysis was performed to evaluate the total carbon (C), hydrogen (H), nitrogen (N), sulfur (S), and oxygen (O) percentages using an Elemental Analyzer based on the standard ASTM D5291, whose results are provided in Table 4, below. The carbon content ranged from 40→45%, with the highest carbon content determined for high-ash paper fraction. The carbon content of various NMSW paper subfractions was similar to two controls representing high cellulose paper and high lignin paper, i.e., hardwood pulp and old corrugated container (OCC), respectively. Hydrogen content ranged from 6.1 to 6.8%, whereas N and S contents were <0.3 and 0.1%, respectively.

TABLE 4
C, H, N, S analysis of various NMSW paper subfractions
(shredded and exposed to steam before extrusion) and
two control feedstocks, i.e., hardwood pulp and OCC.

| | | | | | O %/other |
|---|---|---|---|---|---|
| Sample ID | C % | H % | N % | S % | (diff) |
| #1 Mixed Weight | 41.7 (±0.3) | 6.2 (±0.1) | 0.3 | 0.1 | 51.8 |
| Fraction | | | | | |
| #2 High Lignin | 44.2 (±0.1) | 6.4 (±0.1) | 0.2 | 0.1 | 49.2 |
| #3 High Ash | 45.2 (±0.1) | 6.8 (±0.1) | 0.3 | 0.0 | 47.7 |
| #4 High Cellulose | 40.4 (±0.1) | 6.1 (±0.1) | 0.3 | 0.0 | 53.1 |
| Unwashed | | | | | |
| #5 High Cellulose | 43.5 | 6.5 | 0.3 | 0.0 | 49.7 |
| Washed | | | | | |
| #6 Hardwood Pulp | 42.6 (±0.3) | 6.5 | 0.2 | 0.0 | 50.8 |
| #7 OCC | 45.2 (±0.3) | 6.2 (±0.2) | 0.2 | 0.1 | 48.5 |

The high heating values (HHV) of the various MSW paper subtractions were determined based on ASTM method D240-02. The HHVs of various NMSW paper subfractions are reported in Table 5, below, and ranged from 15.77 MJ/kg to 18.27 MJ/Kg, with an average HHV of 16.53 Mi/Kg.

TABLE 5
HHV results for various NMSW paper subfractions (shredded
and exposed to steam prior to extrusion) and two control
feedstocks, i.e., hardwood pulp and OCC. The analysis
was performed using ASTM method D240-02.

| Sample ID | HHV, MJ/kg |
|---|---|
| #1 Mixed Weight Fraction | 15.77 |
| #2 High Lignin | 17.19 |
| #3 High Ash | 18.27 |
| #4 High Cellulose Unwashed | 15.05 |
| #5 High Cellulose Washed | 16.39 |
| #6 Hardwood Pulp | 16.32 |
| #7 OCC | 17.57 |

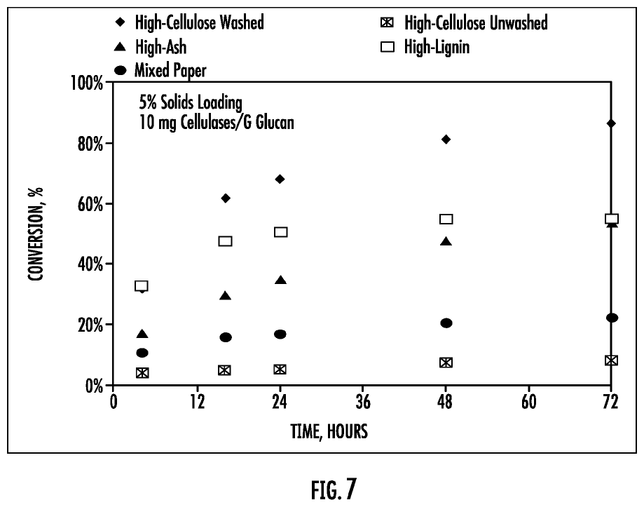

Additionally, homogenized MSW paper subfractions were evaluated for fermentable sugars at 5% solids loading using CTec3 and HTec3 at 8 mg and 2 mg protein/g of glucan, respectively. The glucose and xylose yields obtained after enzymatic hydrolysis are shown in FIG. 7 as a conversion percentage. As expected, the mixed paper sample showed poor susceptibility towards enzymatic hydrolysis, reaching glucose yields of 25% conversion in 72 hours. Interestingly, high-lignin and high-ash subfractions reached 50% conversion. Surprisingly, a high-cellulose unwashed subfraction reached only 12% conversion due to a pH >7. A maximum glucose yield of 88% conversion with a high-cellulose washed subfraction was obtained by mild washing after extrusion.

### Example 7: Extrusion Pretreatment of Food Fraction

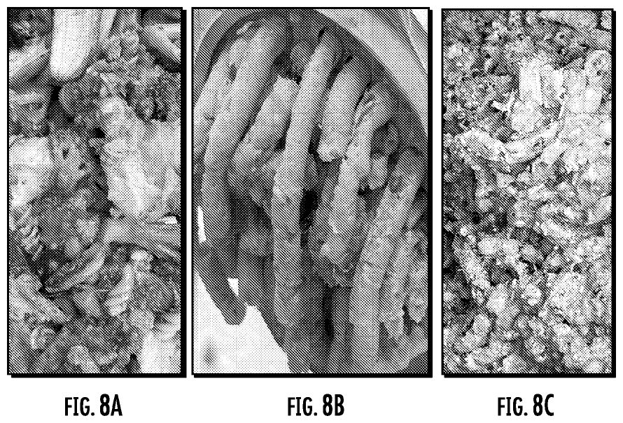

The MSW food fraction was homogenized after sorting the MSW. FIGS. 8A-8C show the MSW food fraction prior to extrusion, exiting the die plate of an extruder extrusion, and collected as a CRF.

### Example 8: Extrusion Pretreatment of Plastic Fraction

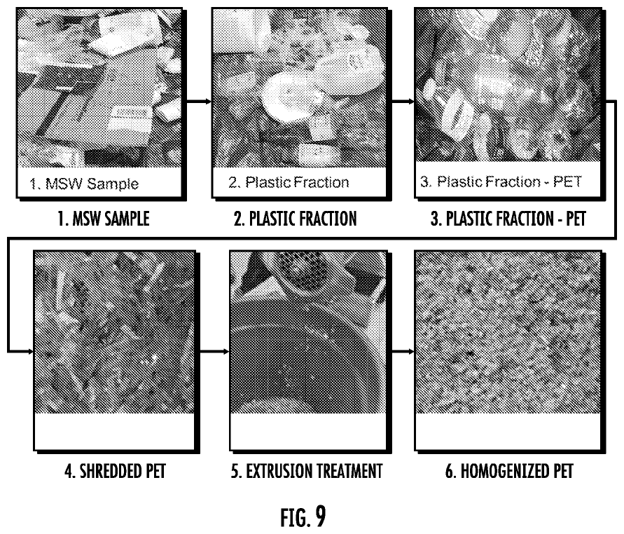

The MSW plastic fraction was obtained by sorting a MSW sample, further sorted to provide a PET subfraction, and then shredded, extruded and homogenized to reduce particle size. FIG. 9 shows exemplary steps for preparing and extruding a PET subfraction.

### Example 9: Extrusion Pretreatment of Textile Fraction

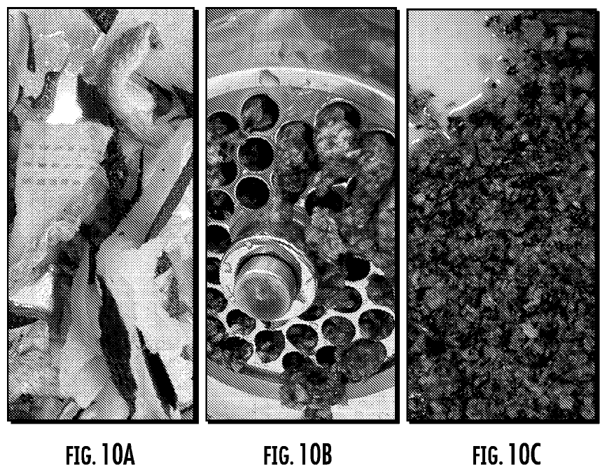

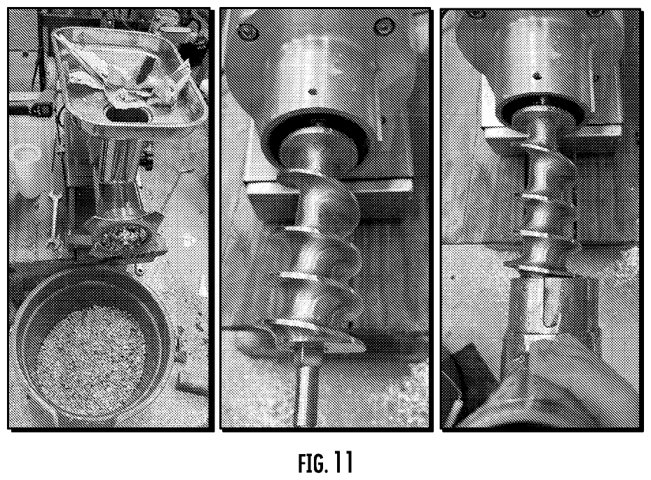

A sample of a MSW textile fraction was water soaked, shredded and homogenized using an extruder to reduce the particle size. See FIGS. 10A-10C.

### Example 10: Production of SAF from NMSW-Derived Sugars

The fermentable sugars from processed MSW paper subfractions can be converted to SAF using a Hydrocarbons (HC) fuel production method via dehydration of the sugar to provide furans, followed by aldol condensation and hydrodeoxygenation (HDO) to produce C9-C16 HC's. See Klein, B., et al., “Techno-Economics Assessment for the Production of HYdrocarbon Fuels via Catalytic Upgrading of Furans,” 2021, National Renewable Energy Lab. (NREL), Golden, Colorado (United States of America). Specifically, furfurals can be produced by sugar dehydration of paper-derived hydrolysates in continuous mode in a tubular flow reactor at a high yield with various hydrothermal, mechanical and biochemical preprocessing to homogenize the feedstock and enhance dehydration. However, no prior work has been attempted on paper waste. It is important to note that the yields of intermediates can be influenced by feedstock variability. The isolated aldol condensation products can then be hydrogenated to stabilize the products prior to more severe conditions for HDO. The hydrogenation can be performed at 100° C. with 68 bars of H₂ and Pd/C catalyst in a tubular flow reactor. HDO can be performed on the H-adducts at 3110° C. for 3 h with 68 bar of H₂ and either a Pd or Pt catalyst on a SiO₂—Al₂O₃ support. The catalyst at 5 wt % of the metal can be made by impregnation of the SiO₂—Al₂O₃ support, which contains 13% Al₂O₃. The freezing point and the cetane number can be measured (ASTM D2887) to understand the jet fuel boiling and diesel fuel boiling range. The heating value of the HC product can be measured and targeted to meet the heating value of gasoline (44-46 MJ/kg) for blending.

### Example 11: Packaging Hand Sheets Making Process from MSW Paper Fraction

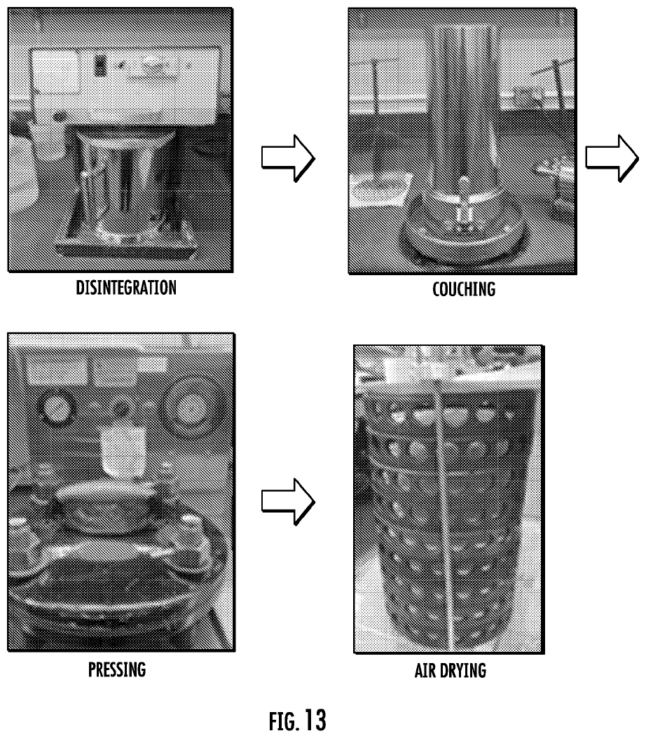

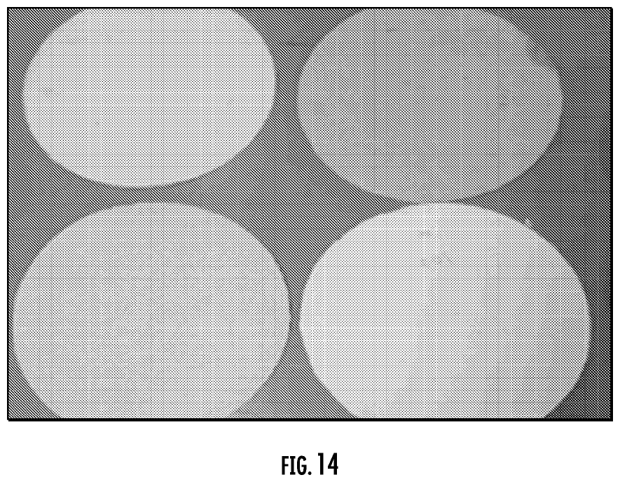

Various MSW paper factions were used for the packaging sheet-making process by following the Tappi T205 method. See FIG. 13. A slurry of preprocessed MSW in water was made at a target consistency of 0.3%. The slurry was disintegrated in a mixer for 10 minutes at 3,000 revolutions per minute. The drainage of the slurry (freeness) was measured according to Tappi T221. Hand sheets were prepared in a British hand sheet mold using standard couching, pressing, and drying conditions. See FIG. 13. The hand sheets were conditioned for 24 hours at 230° C. and 50% relative humidity (RH) and tested for basis weight, caliper, porosity, brightness, tensile force, and stretch. See Table 6, below. Representative images of packaging hand sheets from MSW paper fractions of high ash, high lignin, mixed weight fraction, and high cellulose are shown in FIG. 14. High cellulose content packaging hand sheets showed the highest brightness, tensile force, and stretch. The mixed weight fraction showed lower strength and stretch due to the presence of contaminants.

TABLE 6
Handsheet properties of handsheets prepared from various MSW paper
subfractions (shredded and exposed to steam prior to extrusion).

| | | Basis | | | | Tensile | |
|---|---|---|---|---|---|---|---|
| | | Weight | Caliper | Porosity | Brightness | Force at | Stretch |
| Sample ID | | (gsm) | (micrometer) | (mL) | (%) | Break (N) | (%) |
| #1 Mixed | Avg. | 64.0 | 154.4 | 1.8 | 41.0 | 13.8 | 1.14 |
| Fraction | Std. | 1.4 | 15.4 | 0.1 | 0.4 | 1.6 | 0.35 |
| #2 High | Avg. | 62.4 | 117.1 | 1.5 | 20.2 | 18.8 | 1.81 |
| Lignin | Std. | 1.6 | 3.4 | 0.1 | 0.4 | 1.0 | 0.25 |
| #3 High | Avg. | 61.2 | 139.4 | 2.4 | 54.9 | 15.7 | 1.3 |
| Ash | Std. | 2.5 | 29.8 | 0.9 | 1.4 | 2.9 | 0.34 |
| #4 High | Avg. | 63.4 | 140.0 | 2.0 | 70.6 | 24.0 | 2.1 |
| Cellulose | Std. | 1.9 | 9.4 | 0.0 | 0.5 | 3.3 | 0.65 |

### Example 12: Additional Data from Samples in Table 1B

TABLE 7
Composition of as collected (shredded and extruded without
prior steam or water treatment or soaked (e.g., by
keeping sample in water to soften)) paper subfractions
and food waste of non-recycled MSW (NMSW).

| Sample ID/ | | | | Total | |
|---|---|---|---|---|---|
| Composition | | | | Carbo- | Acid |
| (%) | Glucan | Xylan | Arabinan | hydrate | Insoluble |
| #8 Mixed | 55.4 | 17.5 | 1.7 | 74.6 | 6.6 |
| Weight | | | | (±1.2) | |
| Fraction | | | | | |
| #9 High | 52.9 | 17.2 | 1.8 | 71.9 | 11.6 |
| Lignin | | | | (±0.6) | |
| #10 High Ash | 55.6 | 17.0 | 1.1 | 73.7 | 6.5 |
| | | | | (±1.0) | |
| #11 High | 54.9 | 16.1 | 0.5 | 71.5 | 0.8 |
| Cellulose | | | | (±0.8) | |
| #12 Food | 13.7 | 2.4 | 1.1 | 38.0 | 18.1 |
| Waste | | | | (±0.6)* | |

*Includes whole starch - 14.2%, and extractives free starch - 6.6%; water and ethanol extractives - 23.3%

TABLE 8
C, H, N, S analysis of as collected (shredded and extruded
without prior steam or water treatment or soaked (e.g.,
by keeping the sample in water to soften)) paper subfractions
and food waste of non-recycled MSW (NMSW).

| | | | | | O %/other |
|---|---|---|---|---|---|
| Sample ID | C % | H % | N % | S % | (diff) |
| #8 Mixed Weight | 35.7 | 5.9 | 0.1 | 0.1 | 58.2 |
| Fraction | | | | | |
| #9 High Lignin | 42.6 | 6.3 | 0.2 | 0.1 | 50.8 |
| #10 High Ash | 38.9 | 6.1 | 0.2 | 0.0 | 54.7 |
| #11 High | 35.6 | 5.9 | 0.2 | 0.0 | 58.4 |
| Cellulose | | | | | |
| #12 Food Waste | 42.5 | 6.9 | 4.8 | 0.3 | 45.7 |

TABLE 9
HHV analysis of as collected (shredded and extruded without
prior steam or water treatment or soaked (e.g., by keeping
the sample in water to soften)) paper subfractions and
food waste of non-recycled MSW (NMSW).

| Sample ID | HHV, MJ/kg |
|---|---|
| #8 Mixed Weight Fraction | 15.69 |
| #9 High Lignin | 17.25 |
| #10 High Ash | 15.95 |
| #11 High Cellulose | 13.73 |
| #12 Food Waste | 21.83 |

TABLE 10
Composition of water-homogenized and unwashed (soaked
(e.g., by keeping the sample in water to soften) and
then shredded prior to extrusion) paper subfractions
and textile fraction of non-recycled MSW (NMSW).

| Sample ID/ | | | | Total | |
|---|---|---|---|---|---|
| Composition | | | | Carbo- | Acid |
| (%) | Glucan | Xylan | Arabinan | hydrate | Insoluble |
| #13 Mixed | 52.9 | 15.6 | 1.4 | 69.8 (±1.8) | 14.5 |
| Weight | | | | | |
| Fraction | | | | | |
| #14 High | 56.6 | 16.4 | 1.6 | 74.7 (±4.9) | 15.0 |
| Lignin | | | | | |
| #15 High Ash | 47.1 | 14.9 | 1.1 | 63.1 (±1.0) | 11.9 |
| #16 High | 65.4 | 19.6 | 1.4 | 86.3 (±0.02) | 1.9 |
| Cellulose | | | | | |
| #17 Textile | 41.2 | — | — | 41.2 (±1.2) | 48.2 |

TABLE 11
C, H, N, S of water-homogenized and unwashed (soaked
(e.g., by keeping the sample in water to soften) and
then shredded prior to extrusion) paper subfractions
and textile fraction of non-recycled MSW (NMSW).

| | | | | | O %/other |
|---|---|---|---|---|---|
| Sample ID | C % | H % | N % | S % | (diff) |
| #13 Mixed | 43.4 | 6.3 | 0.2 | 0.0 | 50.1 |
| Weight | | | | | |
| Fraction | | | | | |
| #14 High | 43.5 | 6.5 | 0.2 | 0.1 | 49.8 |
| Lignin | | | | | |
| #15 High Ash | 42.4 | 6.3 | 0.2 | 0.0 | 51.2 |
| #16 High | 41.5 | 6.4 | 0.1 | 0.0 | 51.9 |
| Cellulose | | | | | |
| #17 Textile | 60.5 | 8.1 | 0.7 | 0.1 | 30.7 |

TABLE 12
HHV analysis of water-homogenized and unwashed (soaked
(e.g., by keeping the sample in water to soften) and
then shredded prior to extrusion) paper subfraction
and textile fraction of non-recycled MSW (NMSW).

| Sample ID | HHV, MJ/kg |
|---|---|
| #13 Mixed Weight Fraction | 17.39 |
| #14 High Lignin | 17.29 |
| #15 High Ash | 14.45 |
| #16 High Cellulose | 16.21 |
| #17 Textile | 26.62 |

TABLE 13
Composition of water-homogenized and washed (soaked (e.g., by
keeping the sample in water to soften) and then shredded prior
to extrusion and washed through a vibrating screen after extrusion)
paper subfractions of non-recycled MSW (NMSW).

| Sample ID/ | | | | | |
|---|---|---|---|---|---|
| Composition | | | | Total | Acid |
| (%) | Glucan | Xylan | Arabinan | Carbohydrate | Insoluble |
| #18 Mixed | 64.9 | 17.2 | 0.7 | 82.8 (±1.2) | 6.9 |
| Weight | | | | | |
| Fraction | | | | | |
| #19 High | 63.2 | 16.9 | 1.4 | 81.5 (±1.3) | 10.1 |
| Lignin | | | | | |
| #20 High | 63.8 | 16.9 | 1.4 | 82.1 (±0.13) | 6.2 |
| Ash | | | | | |
| #21 High | 70.2 | 19.5 | 1.2 | 91.0 (±0.78) | 0.5 |
| Cellulose | | | | | |

TABLE 14
C, H, N, S of water-homogenized and washed (soaked (e.g., by
keeping the sample in water to soften) and then shredded prior
to extrusion and washed through a vibrating screen after extrusion)
paper subfractions of non-recycled MSW (NMSW).

| | | | | | O %/other |
|---|---|---|---|---|---|
| Sample ID | C % | H % | N % | S % | (diff) |
| #18 Mixed | 43.6 | 6.6 | 0.3 | 0.1 | 49.6 |
| Weight Fraction | | | | | |
| #19 High Lignin | 43.3 | 6.4 | 0.2 | 0.1 | 50.0 |
| #20 High Ash | 38.6 | 5.6 | 0.2 | 0.0 | 55.5 |
| #21 High | 41.7 | 6.4 | 0.3 | 0.0 | 51.7 |
| Cellulose | | | | | |

TABLE 15
HHV analysis of water-homogenized and washed (soaked (e.g.,
by keeping the sample in water to soften) and then shredded
prior to extrusion and washed through a vibrating screen after
extrusion) paper subfractions of non-recycled MSW (NMSW).

| Sample ID | HHV, MJ/kg |
|---|---|
| #18 Mixed Weight Fraction | 17.20 |
| #19 High Lignin | 17.24 |
| #20 High Ash | 16.64 |
| #21 High Cellulose | 16.16 |

TABLE 16
C, H, N, S analysis of as collected (shredded and extruded
without any prior steam or water treatment or soaked
(e.g., by keeping the sample in water to soften) plastic
fractions of non-recycled MSW (NMSW).

| | | | | | O %/other |
|---|---|---|---|---|---|
| Sample ID | C % | H % | N % | S % | (diff) |
| #22 Plastic all | 72.5 | 9.3 | 0.2 | 0.0 | 18.1 |
| (mixed fraction) | | | | | |
| #23 PET | 61.7 | 4.5 | 0.1 | 0.0 | 33.6 |

TABLE 17
HHV analysis of as collected (shredded and extruded
without any prior steam or water treatment or soaked
(e.g., by keeping the sample in water to soften)
plastic fractions of non-recycled MSW (NMSW).

| Sample ID | HHV, MJ/kg |
|---|---|
| #22 Plastic all | 32.47 |
| (mixed fraction) | |
| #23 PET | 22.93 |

### Example 13: Additional Samples and Data from MSW (NMSW) Collected from Local Municipality Residential Collection Site

Additional samples were collected from a local municipality residential collection site and treated as shown in Table 18, below.

TABLE 18
Sample information and test data from a local municipality residential collection.

| Sample | | | | | | | |
|---|---|---|---|---|---|---|---|
| Type | Processing Steps | | | | | Category | Sample IDs |
| NMSW | NA | NA | Shredded | Extruded | NA | NMSW | #1 As-is- |
| | | | | | | | Sample |
| NMSW | Sorted | NA | Shredded | Extruded | NA | Paper | #2 Whole |
| | | | | | | | Paper |
| | | | | | | | Fraction |
| NMSW | Sorted | NA | Shredded | Extruded | NA | | #3 High |
| | | | | | | | Lignin |
| NMSW | Sorted | NA | Shredded | Extruded | NA | | #4 High Ash |
| NMSW | Sorted | NA | Shredded | Extruded | NA | | #5 High |
| | | | | | | | Cellulose |
| NMSW | Sorted | NA | Shredded | Extruded | NA | | #6 Highly |
| | | | | | | | Contaminated |
| NMSW | Sorted | NA | Shredded | Extruded | NA | Food | #7 Whole |
| | | | | | | | Food Fraction |
| NMSW | Sorted | NA | Shredded | Extruded | NA | Textile | #8 Whole |
| | | | | | | | Textile |
| | | | | | | | Fraction |
| NMSW | Sorted | NA | Shredded | Extruded | NA | Plastic | #9 PET |
| NMSW | Sorted | NA | Shredded | Extruded | NA | | #10 |
| | | | | | | | Polypropylene |
| NMSW | Sorted | NA | Shredded | Extruded | NA | | #11 |
| | | | | | | | Polystyrene |
| NMSW | Sorted | NA | Shredded | Extruded | NA | | #12 Plastic |
| | | | | | | | bags |
| NMSW | Sorted | NA | Shredded | Extruded | NA | | #13 HDPE |

Samples from Table 18 were analyzed as described in the examples above for the samples in Table 1B. Data for the samples from Table 18 is shown in Tables 19-24, below.

TABLE 19
Weight fraction and moisture content
of non-recycled MSW (NMSW).

| | | | Food | | | | |
|---|---|---|---|---|---|---|---|
| MSW Fraction | Paper | Plastic | waste | Textiles | Metal | Glass | Others |
| Weight | 28.4 | 15.5 | 26.3 | 2.4 | 2.6 | 9.0 | 15.8 |
| Fraction (%), | | | | | | | |
| As Collected | | | | | | | |
| Moisture (%) | 18.7 | 0.7 | 58.9 | 13.2 | | | |

TABLE 20
Composition of as collected (shredded and extruded without any
prior steam or water treatment or soaked (e.g., by keeping the
sample in water to soften)) whole NMSW sample, paper subfractions,
food waste, and textile fraction of non-recycled MSW (NMSW).

| Sample ID/ | | | | Total | |
|---|---|---|---|---|---|
| Composition | | | | Carbo- | Acid |
| (%) | Glucan | Xylan | Arabinan | hydrate | Insoluble |
| #1 As-is-Sample | 45.2 | 12.0 | 1.3 | 58.5 | 11.2 |
| #2 Whole Paper | 53.0 | 16.9 | 1.7 | 71.5 | 12.2 |
| Fraction | | | | | |
| #3 High Lignin | 47.4 | 15.5 | 1.8 | 64.6 | 21.0 |
| #4 High Ash | 48.2 | 15.3 | 1.6 | 65.1 | 19.4 |
| #5 High | 64.6 | 18.2 | 1.9 | 84.7 | 3.8 |
| Cellulose | | | | | |
| #6 Highly | 51.9 | 16.4 | 1.7 | 70.0 | 16.5 |
| Contaminated | | | | | |
| #7 Whole Food | 20.3 | 4.6 | 1.9 | 26.8 | 15.0 |
| Fraction | | | | | |
| #8 Whole | 83.8 | 0.0 | 0.0 | 83.8 | 9.9 |
| Textile Fraction | | | | | |

TABLE 21
C, H, N, S analysis of as collected (shredded and extruded without
any prior steam or water treatment or soaked (e.g., by keeping
the sample in water to soften)) whole NMSW sample, paper subfractions,
food waste, and textile fraction of non-recycled MSW (NMSW).

| | | | | | O %/other |
|---|---|---|---|---|---|
| Sample ID | C % | H % | N % | S % | (diff) |
| #1 As-is-Sample | 36.4 | 7.0 | 1.0 | 0.1 | 55.4 |
| #2 Whole Paper | 39.7 | 6.7 | 0.2 | 0.1 | 53.3 |
| Fraction | | | | | |
| #3 High Lignin | 41.2 | 6.6 | 0.2 | 0.1 | 51.9 |
| #4 High Ash | 38.2 | 6.4 | 0.2 | 0.1 | 55.1 |
| #5 High Cellulose | 38.1 | 6.5 | 0.4 | 0.0 | 55.0 |
| #6 Highly | 40.2 | 6.5 | 0.3 | 0.2 | 52.9 |
| Contaminated | | | | | |
| #7 Whole Food | 42.2 | 7.1 | 2.8 | 0.2 | 47.6 |
| Fraction | | | | | |
| #8 Whole Textile | 45.3 | 6.9 | 0.2 | 0.3 | 47.2 |
| Fraction | | | | | |

TABLE 22
HHV analysis of as collected (shredded and extruded without any prior
steam or water treatment or soaked (e.g., by keeping the sample in
water to soften)) whole NMSW sample, various paper subfractions,
food waste, and textile fraction of non-recycled MSW (NMSW).

| Sample ID | HHV, MJ/kg |
|---|---|
| #1 As-is-Sample | 15.22 |
| #2 Whole Paper Fraction | 15.36 |
| #3 High Lignin | 15.38 |
| #4 High Ash | 14.76 |
| #5 High Cellulose | 13.73 |
| #6 Highly Contaminated | 16.02 |
| #7 Whole Food Fraction | 17.46 |
| #8 Whole Textile Fraction | 19.76 |

TABLE 23
C, H, N, S analysis of as collected (shredded and extruded
without any prior steam or water treatment or soaked
(e.g., by keeping the sample in water to soften))
plastic fractions of non-recycled MSW (NMSW).

| | | | | | O %/other |
|---|---|---|---|---|---|
| Sample ID | C % | H % | N % | S % | (diff) |
| #9 PET | 61.0 | 5.0 | 0.1 | 0.0 | 33.9 |
| #10 Polypropylene | 77.4 | 12.6 | 0.2 | 0.0 | 9.8 |
| #11 Polystyrene | 89.3 | 8.5 | 0.2 | 0.0 | 1.9 |
| #12 Plastic bags | 76.7 | 13.8 | 0.8 | 0.0 | 8.7 |
| #13 HDPE | 83.0 | 14.6 | 0.1 | 0.2 | 2.0 |

TABLE 24
HHV analysis of as collected (shredded and extruded
without any prior steam or water treatment or soaked
(e.g., by keeping the sample in water to soften))
plastic fractions of non-recycled MSW (NMSW).

| Sample ID | HHV, MJ/kg |
|---|---|
| #9 PET | 22.91 |
| #10 Polypropylene | 41.07 |
| #11 Polystyrene | 40.19 |
| #12 Plastic bags | 41.69 |
| #13 HDPE | 43.91 |

It will be understood that various details of the presently disclosed subject matter may be changed without departing from the scope of the presently disclosed subject matter. Furthermore, the foregoing description is for the purpose of illustration only, and not for the purpose of limitation.

1. A process for preparing a conversion-ready feedstock (CRF) from a municipal solid waste (MSW), the process comprising:
(a) mechanically homogenizing a MSW; and
(b) performing one or more additional processing steps prior to, during, and/or after step (a), thereby preparing the CRF.

2. The process of claim 1, wherein step (b) comprises removing a hazardous material and/or a noncombustible material from the MSW prior to step (a).

3. The process of claim 1, wherein step (b) comprises sorting a mixed MSW into one or more MSW fractions prior to step (a).

4. The process of claim 1, wherein step (b) comprises shredding the MSW prior to step (a).

5. The process of claim 1, wherein step (b) comprises exposing the MSW to a liquid and/or a gas prior to or during step (a), optionally wherein the liquid and/or gas is steam and/or water.

6. The process of claim 1, wherein step (b) comprises treating the MSW to a treatment selected from the group consisting of a decontamination treatment, a steam treatment, a washing treatment, a screening treatment, a homogenization treatment, a thermal treatment, a thermomechanical treatment, a thermochemical treatment, an AFEX treatment, an anhydrous ammonia treatment, a biological treatment, a biochemical treatment, a bio-thermochemical treatment, a biocatalyst treatment, a chemical catalyst treatment, a photocatalyst treatment, a sonication treatment, a microwave treatment, a photonic treatment, a pyrolysis treatment, a plasma treatment, an electron-beam treatment, an alkali treatment, a dilute acid pretreatment, a subcritical treatment, a supercritical CO2 treatment, a deep eutectic mixture treatment, an enzymatic hydrolysis treatment and any combination thereof, optionally wherein the treatment is performed during step (a).

7. The process of claim 1, wherein step (b) comprises blending MSW with a non-MSW, optionally wherein the blending is performed after step (a) and comprises blending a mechanically homogenized MSW with a non-MSW.

8. The process of claim 1, wherein step (b) comprises performing enzymatic hydrolysis on a mechanically homogenized MSW after step (a).

9. The process of claim 1, wherein step (a) comprises a method selected from the group consisting of extruding, beating, refining, and milling, optionally wherein step (a) comprises extruding.

10. A system for preparing a conversion-ready feedstock (CRF), the system comprising:
(i) a municipal solid waste (MSW); and
(ii) a mechanical homogenization stage configured to receive the MSW from a source and mechanically homogenize the MSW to provide a preprocessed MSW fraction.

11. The system of claim 10, further comprising one or more processing stages configured to process the MSW.

12. The system of claim 10, wherein the mechanical homogenization stage comprises an extruder, optionally wherein the system further comprises a feeding stage configured to collect the MSW in a feed hopper and supply the MSW to the extruder.

13. The system of claim 10, wherein the mechanical homogenization stage comprises a extruder, wherein the extruder comprises a die plate configured to extrude a homogenized MSW in a desired particle size, optionally wherein the die plate is configured at the end of a screw auger configured to move the MSW through the extruding stage.

14. The system of claim 10, wherein the mechanical homogenization stage comprises an extruder comprising a screw auger configured to move the MSW through the extruder, optionally wherein the screw auger is powered and turned by a gear box and a drive mechanism.

15. The system of claim 10, wherein the mechanical homogenization stage comprises a compression zone configured to compress the MSW, optionally wherein the compression zone is present in an extruder configured to extrude a homogenized MSW from a die plate in a desired particle size.

16. The system of claim 10, wherein the mechanical homogenization stage or the one or more processing stages comprise one or more treatment zones, optionally wherein the one or more treatment zones are selected from the group consisting of a steam treatment zone, wherein the steam treatment zone is configured to provide steam to the MSW as it moves through the extruder; an alkaline treatment zone, wherein the alkaline treatment zone is configured to enhance enzymatic hydrolysis of the MSW; and an ammonia fiber expansion (AFEX) treatment zone, wherein the AFEX treatment zone is configured to enhance an enzymatic hydrolysis of the MSW.

17. The system of claim 10, wherein the mechanical homogenization stage comprises an extruder comprising a heating/cooling jacketed barrel configured to surround the MSW as it moves through the extruder.

18. The system of claim 11, wherein the one or more processing stages comprise a stage configured to screen, wash, and/or dry a MSW.

19. The system of claim 11, wherein the one or more processing stages comprise a stage configured to blend a mechanically homogenized MSW provided from a mechanical homogenization stage with a non-MSW.

20. The system of claim 11, wherein the one or more processing stages comprise a stage configured to perform an enzymatic hydrolysis reaction.

21. A conversion-ready feedstock (CRF) wherein the conversion-ready feed stock comprises a fully processed MSW.

22. The CRF of claim 21, wherein the fully processed MSW is a biofuel, optionally wherein the biofuel is selected from the group consisting of a sustainable aviation fuel (SAF), ethanol and a syngas, optionally wherein the syngas comprises a hydrogen fuel.

23. The CRF of claim 21, wherein the fully processed MSW is a packaging, optionally a molded packaging or a flexible packaging.

24. The CRF of claim 21, wherein the fully processed MSW is a fermentable sugar.
